(19) United States
(12) Patent Application Publication
(10) Pub. No.: US 2016/0287374 A1
(43) Pub. Date: Oct. 6, 2016

(54) GRAFT DEVICES AND RELATED SYSTEMS AND METHODS

(71) Applicant: NEOGRAFT TECHNOLOGIES, INC., Taunton, MA (US)

(72) Inventors: Lorenzo SOLETTI, Pittsburgh, PA (US); Cory LEESON, Lakeville, MA (US); Jon MCGRATH, Duxbury, MA (US); Matthew MANNARINO, Waltham, WA (US); John SPIRIDIGLIOZZI, Boston, MA (US)

(21) Appl. No.: 15/036,304

(22) PCT Filed: Nov. 14, 2014
(86) PCT No.: PCT/US2014/065839

§ 371 Date: May 12, 2016

Related U.S. Application Data
(60) Provisional application No. 61/904,807, filed on Nov. 15, 2013.

(51) Int. Cl. A61F 2/07; A61L 27/50; A61F 2/88; A61L 27/34

(52) U.S. Cl. CPC A61F 2/07; A61L 27/34; A61L 27/507; A61F 2/88; A61L 2420/02; A61L 2420/06; A61F 2002/825

(57) ABSTRACT

A graft device for a mammalian patient comprises a tubular conduit and a fiber matrix surrounding the tubular conduit. The fiber matrix can comprise one or more polymers delivered by an electrospinning device. Systems and methods of creating a graft device are also provided.

### RELATED APPLICATIONS

This application claims the benefit of U.S. Provisional Patent Application Ser. No. 61/904,807, filed Nov. 15, 2013, the contents of which are incorporated herein by reference in their entirety. This application is further related to U.S. patent application Ser. No. 12/022,430, filed Jan. 30, 2008; U.S. patent application Ser. No. 13/515,996, filed Jul. 11, 2012; U.S. patent application Ser. No. 13/811,206, filed Jan. 18, 2013; U.S. patent application Ser. No. 13/979,243, filed Jul. 11, 2013; U.S. patent application Ser. No. 13/984,249, filed Aug. 7, 2013; International Patent Application Serial Number PCT/US2012/061790, filed Oct. 25, 2012; International Patent Application Serial Number PCT/US2013/026079, filed Feb. 14, 2013; and International Patent Application Serial Number PCT/US2014/056371, filed Sep. 18, 2014; the contents of each of which are incorporated herein by reference in their entirety.

### TECHNICAL FIELD

This application relates generally to graft devices, and more particularly to graft devices for providing cardiovascular bypass for mammalian patients.

### BACKGROUND

Coronary artery disease, leading to myocardial infarction and ischemia, is a leading cause of morbidity and mortality worldwide. Current treatment alternatives consist of percutaneous transluminal angioplasty, stenting, and coronary artery bypass grafting (CABG). CABG can be carried out using either arterial or venous conduits and is the most effective and most widely used treatment to combat coronary arterial stenosis, with nearly 500,000 procedures being performed annually. In addition, there are approximately 80,000 lower extremity bypass surgeries performed annually. The venous conduit used for bypass procedures is most frequently the autogenous saphenous vein and remains the graft of choice for 95% of surgeons performing these bypass procedures. According to the American Heart Association, in 2004 there were 427,000 bypass procedures performed in 249,000 patients. The long term outcome of these procedures is limited due to occlusion of the graft vein or anastomotic site as a result of intimal hyperplasia (IH), which can occur over a timeframe of months to years.

Development of successful small diameter synthetic or tissue engineered vascular grafts has yet to be accomplished and use of arterial grafts (internal mammary, radial, or gastroepiploic arteries, for example) is limited by the short size, small diameter and availability of these veins. Despite their wide use, failure of arterial vein grafts (AVGs) remains a major problem: 12% to 27% of AVGs become occluded in the first year with a subsequent annual occlusive rate of 2% to 4%. Patients with failed AVGs usually require clinical intervention such as an additional surgery.

IH accounts for 20% to 40% of all AVG failures within the first 5 years after CABG surgery. Several studies have determined that IH develops, to some extent, in all mature AVGs and this development is regarded by many as an unavoidable response of the vein to grafting. IH is characterized by phenotypic modulation, followed by de-adhesion and migration of medial and adventitial smooth muscle cells (SMCs) and myofibroblasts into the intima where they proliferate. In many cases, this response can lead to stenosis and diminished blood flow through the graft. It is thought that IH may be initiated by the abrupt exposure of the veins to the dynamic mechanical environment of the arterial circulation.

### SUMMARY

For these and other reasons, there is a need for systems, methods, and devices which provide enhanced AVGs and other improved grafts for mammalian patients. Desirably, the systems, methods, and devices will improve long term patency and reduce (e.g., minimize) surgical and device complications such as those caused by kinking of graft devices.

Embodiments of the present inventive concepts can be directed to graft devices for mammalian patients, as well as systems and methods for producing these graft devices.

In some aspects of the technology, a graft device comprises a tubular conduit and a fiber matrix surrounding the tubular conduit.

In some embodiments, the fiber matrix comprises at least one thermoplastic co-polymer. The fiber matrix can comprise a first material and a second different material. The second material can comprise a softer material than the first material. The fiber matrix can comprise relatively equal amounts of the first material and the second material. The second material can comprise polydimethylsiloxane and a polyether-based polyurethane. The first material can comprise aromatic methylene diphenyl isocyanate.

In some embodiments, the fiber matrix comprises a material selected from the group consisting of: polymer selected from the group consisting of: polyolefins; polyurethanes; polyvinylchlorides; polyamides; polyimides; polyacrylates; polyphenolics; polystyrene; polycaprolactone; polylactic acid; polyglycolic acid; and combinations thereof. The fiber matrix can comprise a polymer applied to the tubular conduit when dissolved in a solvent.

In some embodiments, the fiber matrix comprises a material selected from the group consisting of: polyglycerol sebacate; hyaluric acid; silk fibroin collagen; elastin; poly(p-dioxanone); poly(3-hydroxybutyrate); poly(3-hydroxyvalerate); poly(valcrolactone); poly(tartronic acid); poly(beta-malonic acid); poly(propylene fumarates); a polyanhydride; a tyrosine-derived polycarbonate; a polyorthoester; a degradable polyurethane; a polyphosphazene; and combinations thereof.

In some embodiments, the fiber matrix comprises a thermoplastic comprising at least two materials. The thermoplastic can comprise a first material and a second material, and the first material can be softer than the second material. The first material can comprise segments including polydimethylsiloxane and polyhexamethylene oxide, and the second material can comprise segments including aromatic methylene diphenyl isocyanate.

In some embodiments, the fiber matrix comprises a material applied with a device selected from the group consisting of: an electrospinning device; a melt-spinning device; a melt-electrospinning device; a misting assembly; a sprayer; an electrosprayer; a three-dimensional printer; and combinations thereof.

In some embodiments, the fiber matrix comprises a thickness between 100 μm and 1000 μm. The fiber matrix can comprise a thickness between 150 μm and 400 μm. The fiber matrix can comprise a thickness between 220 μm and 280 μm. The fiber matrix can comprise a thickness of approximately 250 μm.

In some embodiments, the fiber matrix comprises an inner layer and a surrounding outer layer. The device can further comprise a kink resisting element positioned between the inner layer and the outer layer. The kink resisting element can comprise a spine.

In some embodiments, the fiber matrix comprises fibers with an average diameter of at least 5 μm.

In some embodiments, the fiber matrix comprises fibers with an average diameter between 6 μm and 15 μm. The fiber matrix can comprise fibers with an average diameter of approximately 7.8 μm. The fiber matrix can comprise fibers with an average diameter of approximately 8.6 μm.

In some embodiments, the fiber matrix comprises an average porosity between 40% and 80%. The fiber matrix can comprise an average porosity of approximately 50.4%. The fiber matrix can comprise an average porosity of approximately 46.9%.

In some embodiments, the fiber matrix comprises a compliance between 0.2×10⁻⁴/mmHg and 3.0×10⁴/mmHg.

In some embodiments, the fiber matrix comprises an average elastic modulus between 10 Pa and 18 MPa.

In some embodiments, the fiber matrix comprises a property selected from the group consisting of: stress measured at 5% strain comprising between 0.4 MPa and 1.1 MPa; ultimate stress of 4.5 MPa to 7.0 MPa; ultimate strain of 200% to 400%; and combinations thereof.

In some embodiments, the fiber matrix comprises a property selected from the group consisting of: stress at 5% strain comprising between 0.6 MPa and 1.3 MPa; ultimate stress of 5.0 MPa to 7.5 MPa; ultimate strain of 200% to 400%; and combinations thereof.

In some embodiments, the fiber matrix comprises an average compliance of approximately 0.2×10⁻⁴/mmHg to 3.0×10⁻⁴/mmHg.

In some embodiments, the fiber matrix comprises an average circumferential elastic modulus of between 10 MPa and 15 MPa.

In some embodiments, the fiber matrix comprises an average circumferential elastic modulus of between 12 MPa and 18 MPa.

In some embodiments, the fiber matrix is constructed and arranged to provide a suture retention strength of at least one of between 2.0 N and 4.0 N with 6-0 Prolene suture or between 1.5 N and 3.0 N with 7-0 Prolene suture.

In some embodiments, the fiber matrix is constructed and arranged to provide a suture retention strength of at least one of between 2.3 N and 4.3 N with 6-0 Prolene suture or between 2.0 N and 3.5 N with 7-0 Prolene suture.

In some embodiments, the device further comprises a kink resisting element. The kink resisting element can be positioned between the tubular conduit and the fiber matrix. The fiber matrix can comprise an inner layer and an outer layer, and the kink resisting element can be positioned between the fiber matrix inner layer and outer layer. The fiber matrix can comprise a first thickness and the inner layer can comprise a second thickness approximately between 1% and 99% of the first thickness. The second thickness can comprise a thickness approximately between 25% and 60% of the first thickness. The second thickness can comprise a thickness of approximately 33% of the first thickness. The kink resisting element can comprise a spine. The spine can comprise a first support portion and a second support portion, and at least one of the first support portion or the second support portion can be constructed and arranged to rotate relative to the other to receive the tubular conduit. The spine can comprise a first support portion comprising a first set of projections, and a second support portion comprising a second set of projections, and the first set of projections can interdigitate with the second set of projections. The interdigitating projections can be spaced approximately 0.125 inches from each other. The interdigitating projections can comprise a series of overlapping distal ends. The overlapping distal ends can overlap at least 2.5 mm. The kink resisting element can comprise at least one filament with a diameter between 0.4 mm and 0.5 mm. The kink resisting element can comprise a resiliently biased element. The kink resisting element can be resiliently biased with a heat treatment. The kink resisting element can comprise a surface treated element. The kink resisting element surface treatment can increase surface roughness of the kink resisting element.

In some embodiments, the tubular conduit comprises a varying circumferential shape.

In some embodiments, the tubular conduit comprises harvested tissue. The tubular conduit can comprise tissue selected from the group consisting of: saphenous vein; vein; artery; urethra; intestine; esophagus; ureter; trachea; bronchi; duct; fallopian tube; and combinations thereof.

In some embodiments, the tubular conduit comprises artificial material. The tubular conduit can comprise artificial material selected from the group consisting of: polytetrafluoroethylene (PTFE); expanded PTFE (ePTFE); polyester; polyvinylidene fluoride/hexafluoropropylene (PVDF-HFP); silicone; polyethylene; polypropylene; polyester-based polymer; polyether-based polymer; thermoplastic rubber; and combinations thereof.

In some aspects of the technology, a system for applying a fiber matrix to a tubular conduit to create a graft device comprises a rotating assembly; a polymer delivery assembly; and a controller. The rotating assembly can be constructed and arranged to rotate the tubular conduit. The polymer delivery assembly can be constructed and arranged to deliver the fiber matrix to the tubular conduit. The controller can be constructed and arranged to control the polymer delivery assembly and the rotating assembly.

In some embodiments, the system further comprises a polymer for delivery by the polymer delivery assembly. The polymer can comprise at least one thermoplastic co-polymer. The polymer can comprise at least two materials. The polymer can comprise a first material and a second material, and the first material can be softer than the second material. The fiber matrix can comprise relatively equal amounts of the first material and the second material. The first material can comprise segments including polydimethylsiloxane and polyhexamethylene oxide, and the second material can comprise segments including aromatic methylene diphenyl isocyanate. The system can further comprise a solvent combined with the polymer prior to application of the polymer to the tubular conduit. The solvent can comprise HFIP. The solvent can comprise 99.97% minimum purity HFIP. The solvent can comprise a weight to volume ratio of concentration between 24.5 and 25.5% grams/mL. The polymer can comprise molecular weight distribution between 80,000 and 150,000. The polymer can comprise a viscosity between 2000 cP and 24000 when measured at 25° C. and with a shear rate of 2020 s⁻¹. The polymer can comprise a conductivity between 0.40/cm and 1.7 μS/cm when measured at a temperature between 20° C. and 22° C. The polymer can comprise a surface tension between 21.5 mN/m and 23.0 mN/m when measured at approximately 25° C.

In some embodiments, the polymer delivery assembly comprises an electrospinning device.

In some embodiments, the system is constructed and arranged to deliver polymer at a flow rate of between 10 ml/hr and 25 ml/hr. The system can be constructed and arranged to deliver polymer at a flow rate of approximately 15 ml/hr. The system can be constructed and arranged to deliver polymer at a flow rate of approximately 20 ml/hr.

In some embodiments, the polymer delivery assembly comprises a nozzle including an orifice constructed and arranged to deliver the fiber matrix to the tubular conduit. The system can be constructed and arranged such that the tubular conduit is positioned approximately 12.2 cm and 12.8 cm from the orifice during application of the fiber matrix. The system can be constructed and arranged to translate the nozzle a distance of approximately 29 cm in one direction. The system can be constructed and arranged to translate the nozzle at a linear rate of between 40 mm/sec and 150 mm/sec. The system can be constructed and arranged to translate the nozzle at a linear rate of between 50 mm/sec and 80 mm/sec. The system can be constructed and arranged to translate the nozzle at a linear rate of between 55 mm/sec and 65 mm/sec. The system can be constructed and arranged to translate the nozzle at a linear rate of approximately 60 mm/sec. The system can be constructed and arranged to translate the nozzle in a reciprocal motion. The system can be constructed and arranged to rapidly change direction at the end of each reciprocating linear travel. The system can be constructed and arranged to apply a voltage of approximately +15 kV to the nozzle. The system can be constructed and arranged to apply a voltage of approximately +17 kV to the nozzle. The nozzle can comprise 304 stainless steel. The nozzle can comprise a hypotube. The nozzle can comprise a length of approximately 1.0 inches. The nozzle can comprise an inner diameter of approximately 0.016 inches. The nozzle can comprise a wall thickness between 0.004 inches and 0.018 inches. The nozzle can comprise a wall thickness of approximately 0.006 inches. The nozzle can comprise a central axis and a blunt end tip relatively orthogonal to the central axis. The system can be constructed and arranged to maintain an object-free space distal to the orifice. In some embodiments, the object-free space is void of any objects that contain a charge and/or are electrically conductive. In some embodiments, the object-free space is void of any objects that may be in a location that would interfere with the flight path of a fiber traveling between a nozzle and the tubular conduit (e.g. to prevent deposition of a fiber on an undesired object). The object-free space can comprise a diameter of at least 10 cm. The nozzle can comprise a proximal portion and wherein the polymer delivery assembly can further comprise a sleeve surrounding the proximal portion of the nozzle. The sleeve can comprise an insulating sleeve. The sleeve can comprise a distal end positioned approximately 1 cm from the orifice. The nozzle can be offset from a central axis of the tubular conduit during application of the fiber matrix. The offset comprises a distance of between 0.5 cm and 0.8 cm. The polymer delivery assembly can further comprise a sheath surrounding the nozzle. The sheath can be constructed and arranged to reduce icicle formation. The sheath can comprise a distal end positioned flush with the orifice. The polymer delivery assembly can comprise a gap positioned between the sheath and the nozzle. The sheath can comprise a dimension selected from the group consisting of: length of approximately 16 mm; ID of approximately 0.08 inches; OD of approximately 0.118 inches; wall thickness between 0.025 inches and 0.085 inches; wall thickness approximately 0.055 inches; and combinations thereof. The nozzle can comprise a proximal portion and wherein the polymer delivery assembly can further comprise a sleeve surrounding the proximal portion of the nozzle. The sheath can comprise a proximal portion and wherein the sleeve further surrounds the proximal portion of the sheath.

In some embodiments, the system is constructed and arranged to deliver the fiber matrix during a cumulative application time period. The cumulative application time period can comprise approximately 11 minutes and 40 seconds for a fiber matrix with a diameter between 3.4 mm and 4.2 mm. The cumulative application time period can comprise approximately 14 minutes and 0 seconds for a fiber matrix with a diameter between 4.2 mm and 5.1 mm. The cumulative application time period can comprise approximately 17 minutes and 30 seconds for a fiber matrix with a diameter between 5.1 mm and 6.0 mm. The cumulative application time period can comprise approximately 9 minutes and 30 seconds for a fiber matrix with a diameter between 3.4 mm and 4.2 mm. The cumulative application time period can comprise approximately 11 minutes and 30 seconds for a fiber matrix with a diameter between 4.2 mm and 5.1 mm. The cumulative application time period can comprise approximately 13 minutes and 40 seconds for a fiber matrix with a diameter between 5.1 mm and 6.0 mm.

In some embodiments, the polymer delivery assembly is constructed and arranged to deliver a fiber matrix comprising an inner layer and an outer layer. The system can be constructed and arranged for a kink resisting element to be positioned between the inner layer and the outer layer. The system can be constructed and arranged to deliver the fiber matrix during a cumulative application time period, and the inner layer can be applied during approximately one-third of the cumulative application time period and the outer layer can be applied during approximately two-thirds of the cumulative application time period.

In some embodiments, the system further comprises a mandrel constructed and arranged for insertion into the tubular conduit. The system can be constructed and arranged to apply a voltage of approximately −2 kV to the mandrel. The system can be constructed and arranged to rotate the mandrel at a velocity between 100 rpm and 400 rpm. The system can be constructed and arranged to rotate the mandrel at a velocity between 200 rpm and 300 rpm. The system can be constructed and arranged to rotate the mandrel at a velocity between 240 pm and 260 rpm. The system can be constructed and arranged to rotate the mandrel at a velocity of approximately 250 rpm. The mandrel can comprise a material selected from the group consisting of: 304 stainless steel; 316 stainless steel; and combinations thereof. The mandrel can comprise a surface finish of approximately Ra=0.1-0.8 μm. The mandrel can comprise a length less than or equal to 45cm. The mandrel can comprise a length of at least 30 cm. The mandrel can comprise one or more mandrels with a diameter selected from the group consisting of: 3.0 mm; 3.5 mm; 4.0 mm; 4.5 mm; and combinations thereof. The mandrel can comprise a first mandrel and a second mandrel. The first mandrel can comprise a first diameter and the second mandrel can comprise a second diameter different than the first diameter. The mandrel can comprise a mandrel central axis and the nozzle can comprise a nozzle central axis positioned relatively orthogonal to the mandrel central axis during application of the fiber matrix. The mandrel can comprise a mid portion and the system can be constructed and arranged to maintain an object-free space surrounding at least the mid portion of the nozzle. The object-free space can comprise a diameter of at least 10 cm.

In some embodiments, the system further comprises an environmental control assembly constructed and arranged to control at least one environmental parameter proximate the polymer delivery assembly and/or the tubular conduit during the application of the fiber matrix. The environmental control assembly can comprise a component selected from the group consisting of: a fan; a source of a gas such as a dry compressed air source; a source of gas at a negative pressure; a vapor source such as a source including a buffered vapor, an alkaline vapor and/or an acidic vapor; a filter such as a HEPA filter; a dehumidifier; a humidifier; a heater; a chiller; and electrostatic discharge reducing ion generator; and combinations thereof. The at least one environmental parameter can comprise humidity level. The environmental control assembly can be constructed and arranged to provide flow of gas proximate the polymer delivery assembly and/or tubular conduit during application of the fiber matrix. The flow of gas can comprise flow of gas comprising less than or equal to 2 ppm water content. The flow of gas can be constructed and arranged to remove solvent vapors. The flow of gas can be constructed and arranged to remove HFIP vapors. The flow of gas can be constructed and arranged to replace gas surrounding the polymer delivery assembly and/or tubular conduit at least once every 3 minutes. The flow of gas can be constructed and arranged to replace gas surrounding the polymer delivery assembly and/or tubular conduit at least once every 1 minute. The flow of gas can be constructed and arranged to replace gas surrounding the polymer delivery assembly and/or tubular conduit at least once every 30 seconds. The environmental control assembly can comprise a filter constructed and arranged to filter gas outflow from the environmental control assembly. The environmental control assembly can be constructed and arranged to provide an initial flow of gas at a flow rate of at least 30 L/min. The environmental control assembly can be constructed and arranged to provide an initial flow of gas at a flow rate of at least 60 L/min. The environmental control assembly can be constructed and arranged to provide a reduced flow of gas after the initial flow of gas, and the reduced flow of gas can be provided at a flow rate between 10 L/min and 30 L/min. The at least one environmental parameter can comprise a temperature level. The environmental control chamber can be constructed and arranged to maintain a temperature between 15° C. and 25° C. The environmental control chamber can be constructed and arranged to maintain a temperature between 16° C. and 20° C. The environmental control chamber can be further constructed and arranged to maintain a relative humidity less than or equal to 24%.

In some embodiments, the system further comprises a tubular conduit drying assembly. The tubular conduit drying assembly can comprise gauze.

In some embodiments, the system further comprises a modification assembly. The modification assembly can comprise a component selected from the group consisting of; a robotic device such as a robotic device configured to apply a spine to a tubular conduit; a nozzle, such as a nozzle configured to deliver an agent; an energy delivery element such as a laser delivery element such as a laser excimer diode or other element configured to trim one or more components of a graft device; a fluid jet such as a water jet or air jet configured to deliver fluid during the application of the fiber matrix to the tubular conduit; a cutting element such as a cutting element configured to trim a spine and/or the fiber matrix; a mechanical abrader; and combinations thereof. The modification assembly can be constructed and arranged to modify the tubular conduit. The modification assembly can be constructed and arranged to modify the fiber matrix. The system can further comprise a kink resisting element, and the modification assembly can be constructed and arranged to modify the kink resisting element. The system can further comprise a kink resisting element, and the modification assembly can be constructed and arranged to apply the kink resisting element about the tubular conduit. The modification assembly can be constructed and arranged to deliver an agent. The agent (e.g. a wax or other protective covering material) can be constructed and arranged to be delivered to the tubular conduit to prevent damage to the tubular conduit by a solvent. The agent can comprise an adhesive delivered between the tubular conduit and the fiber matrix.

In some embodiments, the fiber matrix comprises at least one thermoplastic co-polymer. The fiber matrix can comprise a first material and a second different material. The second material can comprise a softer material than the first material. The fiber matrix can comprise relatively equal amounts of the first material and the second material. The second material can comprise polydimethylsiloxane and a polyether-based polyurethane. The first material can comprise aromatic methylene diphenyl isocyanate.

In some embodiments, the fiber matrix comprises a material selected from the group consisting of: polymer selected from the group consisting of: polyolefins; polyurethanes; polyvinylchlorides; polyamides; polyimides; polyacrylates; polyphenolics; polystyrene; polycaprolactone; polylactic acid; polyglycolic acid; and combinations thereof. The fiber matrix can comprise a polymer applied to the tubular conduit when dissolved in a solvent. The solvent can comprise a material selected from the group consisting of: hexafluoroisopropanol (HFIP); acetone; methyl ethyl ketone; benzene; toluene; xylene; dimethyleformamide; dimethylacetamide; propanol; ethanol; methanol; propylene glycol; ethylene glycol; trichloroethane; trichloroethylene; carbon tetrachloride; tetrahydrofuran; cyclohexone; cyclohexpropylene glycol; DMSO; tetrahydrofuran; chloroform; methylene chloride; and combinations thereof.

In some embodiments, the fiber matrix comprises a material selected from the group consisting of: polyglycerol sebacate; hyaluric acid; silk fibroin collagen; elastin; poly(p-dioxanone); poly(3-hydroxybutyrate); poly(3-hydroxyvalerate); poly(valcrolactone); poly(tartronic acid); poly(beta-malonic acid); poly(propylene fumarates); a polyanhydride; a tyrosine-derived polycarbonate; a polyorthoester; a degradable polyurethane; a polyphosphazene; and combinations thereof.

In some embodiments, the fiber matrix comprises a thermoplastic comprising at least two materials. The thermoplastic can comprise a first material and a second material, and the first material can be softer than the second material. The first material can comprise segments including polydimethylsiloxane and polyhexamethylene oxide, and the second material can comprise segments including aromatic methylene diphenyl isocyanate.

In some embodiments, the fiber matrix comprises a material applied with a device selected from the group consisting of: an electrospinning device; a melt-spinning device; a melt-electrospinning device; a misting assembly; a sprayer; an electrosprayer; a three-dimensional printer; and combinations thereof.

In some embodiments, the fiber matrix comprises a thickness between 100 μm and 1000 μm. The fiber matrix can comprise a thickness between 150 μm and 400m. The fiber matrix can comprise a thickness between 220 μm and 280 μm. The fiber matrix can comprise a thickness of approximately 250 μm.

In some embodiments, the fiber matrix comprises an inner layer and a surrounding outer layer. The device can further comprise a kink resisting element positioned between the inner layer and the outer layer. The kink resisting element can comprise a spine.

In some embodiments, the fiber matrix comprises fibers with an average diameter of at least 5 μm .

In some embodiments, the fiber matrix comprises fibers with an average diameter between 6 μm and 15 μm. The fiber matrix can comprise fibers with an average diameter of approximately 7.8 μm. The fiber matrix can comprise fibers with an average diameter of approximately 8.6 μm.

In some embodiments, the fiber matrix comprises an average porosity between 40% and 80%. The fiber matrix can comprise an average porosity of approximately 50.4%. The fiber matrix can comprise an average porosity of approximately 46.9%.

In some embodiments, the fiber matrix comprises a compliance between 0.2×10⁴/mmHg and 3.0×10⁻⁴/mmHg.

In some embodiments, the fiber matrix comprises an average elastic modulus between 10 MPa and 18 MPa.

In some embodiments, the fiber matrix comprises a property selected from the group consisting of: stress measured at 5% strain comprising between 0.4 MPa and 1.1 MPa; ultimate stress of 4.5 MPa to 7.0 MPa; ultimate strain of 200% to 400%; and combinations thereof.

In some embodiments, the fiber matrix comprises a property selected from the group consisting of: stress at 5% strain comprising between 0.6 MPa and 1.3 MPa; ultimate stress of 5.0 MPa to 7.5 MPa; ultimate strain of 200% to 400%; and combinations thereof.

In some embodiments, the fiber matrix comprises an average compliance of approximately 0.2×10⁴/mmHg to 3.0×10⁻⁴/mmHg.

In some embodiments, the fiber matrix comprises an average circumferential elastic modulus of between 10 MPa and 15 MPa.

In some embodiments, the fiber matrix comprises an average circumferential elastic modulus of between 12 MPa and 18 MPa.

In some embodiments, the fiber matrix is constructed and arranged to provide a suture retention strength of at least one of between 2.0N and 4.0N with 6-0 Prolene suture or between 1.5N and 3.0N with 7-0 Prolene suture.

In some embodiments, the fiber matrix is constructed and arranged to provide a suture retention strength of at least one of between 2.3 N and 4.3 N with 6-0 Prolene suture or between 2.0 N and 3.5 N with 7-0 Prolene suture.

In some embodiments, the system further comprises a kink resisting element. The kink resisting element can be positioned between the tubular conduit and the fiber matrix. The fiber matrix can comprise an inner layer and an outer layer, and the kink resisting element can be positioned between the fiber matrix inner layer and outer layer. The fiber matrix can comprise a first thickness and the inner layer can comprise a second thickness approximately between 1% and 99% of the first thickness. The second thickness can comprise a thickness approximately between 25% and 60% of the first thickness. The second thickness can comprise a thickness of approximately 33% of the first thickness. The kink resisting element can comprise a spine. The spine can comprise a first support portion and a second support portion, and at least one of the first support portion or the second support portion can be constructed and arranged to rotate relative to the other to receive the tubular conduit. The spine can comprise a first support portion comprising a first set of projections, and a second support portion comprising a second set of projections, and the first set of projections can interdigitate with the second set of projections. The interdigitating projections can be spaced approximately 0.125 inches from each other. The interdigitating projections can comprise a series of overlapping distal ends. The overlapping distal ends can overlap at least 2.5 mm. The kink resisting element can comprise at least one filament with a diameter between 0.4 mm and 0.5 mm. The kink resisting element can comprise a resiliently biased element. The kink resisting element can be resiliently biased with a heat treatment. The kink resisting element can comprise a surface treated element. The kink resisting element surface treatment can increase surface roughness of the kink resisting element.

In some embodiments, the device is constructed and arranged to be positioned in an in-vivo geometry including at least one arced portion comprising a radius of curvature of as low as 0.5 cm.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same or like elements. The drawings are not necessarily to scale, emphasis instead being placed upon illustrating the principles of the preferred embodiments.

FIG. 1 is a side, partial cutaway view of an example graft device, consistent with the present inventive concepts.

FIG. 2A is a sectional view of an example graft device of FIG. 1, comprising a tubular conduit and a surrounding fiber matrix, consistent with the present inventive concepts.

FIG. 2B is a sectional view of another example graft device of FIG. 1, comprising a tubular conduit, a spine and a surrounding fiber matrix, consistent with the present inventive concepts.

FIG. 3 is a schematic view of an example system for producing a graft device with an electrospun fiber matrix, consistent with the present inventive concepts.

FIG. 4 is a side sectional view of a portion of the electrospinning device of FIG. 3, consistent with the present inventive concepts.

### DETAILED DESCRIPTION

The terminology used herein is for the purpose of describing particular example embodiments and is not intended to be limiting of the inventive concepts. Furthermore, embodiments of the present inventive concepts may include several novel features, no single one of which is solely responsible for its desirable attributes or which is essential to practicing an inventive concept described herein. As used herein, the singular forms “a,” “an” and “the” are intended to include the plural forms as well, unless the context clearly indicates otherwise.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element or intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g., rotated 90 degrees or at other orientations) and the spatially relative descriptors used herein interpreted accordingly.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

Provided herein are graft devices for implantation in a mammalian patient, such as to carry fluids (e.g. blood or other body fluid) from a first anatomical location to a second anatomical location. The graft devices include a tubular conduit, such as a harvested blood vessel segment, other harvested tissue or an artificial conduit, and a fiber matrix that surrounds the tubular conduit. The fiber matrix is typically applied with one or more of: an electrospinning device; a melt-spinning device; a melt-electrospinning device; a misting assembly; a sprayer; an electrosprayer; a fuse deposition device; a selective laser sintering device; a three-dimensional printer; or other fiber matrix delivery device. The fiber matrix delivery process can be performed in an operating room, such as when the tubular conduit is a harvested saphenous vein segment to be anastomosed between the aorta and a location on a diseased coronary artery distal to an occlusion. In these cardiovascular bypass procedures, end to side anastomotic connections are typically used to attach the graft device to the aorta and the diseased artery. Alternatively, a side to side anastomosis can be used, such as to attach an end of the graft device to multiple arteries in a serial fashion.

The fiber matrix can comprise one or more materials, such as one or more similar or dissimilar polymers as described in detail herebelow. The fiber matrix can comprise a biodegradable or bioerodible (hereinafter “biodegradable”) material or otherwise be configured such that the support to the graft device changes over time after implantation. Numerous biodegradable polymers can be used such as: polylactide, poylglycolide, polysaccharides, proteins, polyesters, polyhydroxyal kanoates, polyalkelene esters, polyamides, polycaprolactone, polyvinyl esters, polyamide esters, polyvinyl alcohols, polyanhydrides and their copolymers, modified derivatives of caprolactone polymers, polytrimethylene carbonate, polyacrylates, polyethylene glycol, hydrogels, photo-curable hydrogels, terminal diols, and combinations of these. Dunn et al. (U.S. Pat. No. 4,655,777) discloses a medical implant including bioabsorbable fibers that reinforce a bioabsorbable polymer matrix. Alternatively or additionally, the fiber matrix can comprise one or more portions including durable or otherwise non-biodegradable materials configured to remain intact for long periods of time when implanted, such as at least 6 months or at least 1 year.

The graft devices can further include a spine or other kink resisting element, such as to prevent luminal narrowing, radial collapse, kinking and/or other undesired movement of the graft device (e.g. movement into an undesired geometric configuration), such as while implanting the graft device during a surgical procedure and/or at a time after implantation. The spine can be placed inside the tubular conduit, between the tubular conduit and the fiber matrix, between layers or within layers of the fiber matrix and/or outside the fiber matrix. The spine can comprise a biodegradable material or otherwise be configured to provide a temporary support to the graft device. Alternatively or additionally, the spine can comprise one or more portions including durable or otherwise non-biodegradable materials configured to remain intact for long periods of time when implanted, such as at least 6 months or at least 1 year.

Also provided herein are systems and methods for producing a graft device comprising a conduit and a surrounding fiber matrix. Systems typically include an electrospinning device and/or other fiber or fiber matrix delivering assembly. In some embodiments, the graft device further comprises a spine or other kink resisting element. The spine can comprise a component that is applied, placed and/or inserted, such as by the fiber matrix delivery assembly (e.g. automatically or semi-automatically) or with a placement or insertion tool (e.g. manually).

Devices of the present invention can include an electrospun fiber matrix such as those disclosed in U.S. patent application Ser. No. 13/502,759, filed Apr. 19, 2012, the contents of which are incorporated herein by reference in their entirety. The present invention includes graft devices, as well as systems, tools and methods for producing graft devices, such as those disclosed in applicant's co-pending applications U.S. patent application Ser. No. 13/515,996, filed Jul. 11, 2012; U.S. patent application Ser. No. 13/811,206, filed Jan. 18, 2013; U.S. patent application Ser. No. 13/979,243, filed Jul. 11, 2013; U.S. patent application Ser. No. 13/984,249, filed Aug. 7, 2013; International Patent Application Ser. No. PCT/US2012/061790, filed Oct. 25, 2012; International Patent Application Ser. No. PCT/US2013/026079, filed Feb. 14, 2013; the contents of each of which are incorporated herein by reference in their entirety.

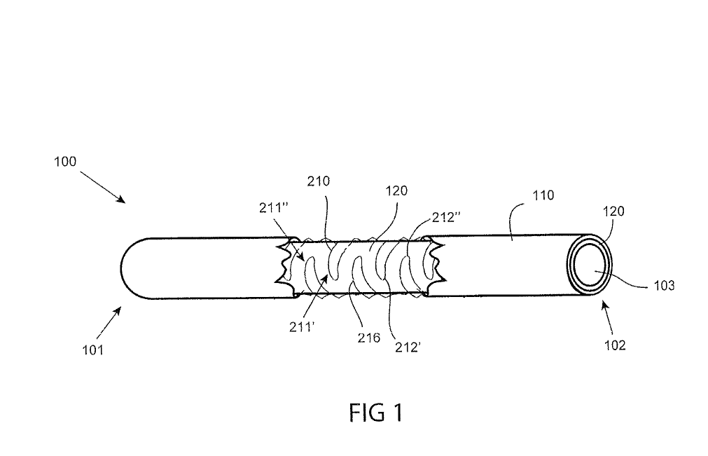

Referring now to FIG. 1, a side, partial cut-away view of a graft device is illustrated, consistent with the present inventive concepts. Graft device 100 includes tubular conduit 120 and fiber matrix 110. In some embodiments, graft device 100 further includes spine 210 as shown. Tubular conduit 120 is circumferentially surrounded by fiber matrix 110. Graft device 100 includes a first end 101 and a second end 102, and is preferably configured to be placed between a first body location and a second body location of a patient. Graft device 100 includes lumen 103 from first end 101 to second end 102, such as to carry blood or other fluid when graft device 100 is connected between two body locations, such as between two blood vessels in a cardiovascular bypass procedure.

Tubular conduit 120 can comprise a varying circumferential shape (e.g. a varying diameter of its outer surface), and fiber matrix 110 and/or spine 210 can be constructed and arranged to conform to the varying circumferential shape of conduit 120. Conduit 120 can comprise harvested tissue, such as a segment of a harvested vessel, such as a saphenous vein or other vein. In some embodiments, conduit 120 comprises tissue selected from the group consisting of: saphenous vein; vein; artery; urethra; intestine; esophagus; ureter; trachea; bronchi; duct; fallopian tube; and combinations of these. Alternatively or additionally, conduit 120 can comprise artificial material, such as a material selected from the group consisting of: polytetrafluoroethylene (PTFE); expanded PTFE (ePTFE); polyester; polyvinylidene fluoride/hexafluoropropylene (PVDF-HFP); silicone; polyethylene; polypropylene; polyester-based polymer; polyether-based polymer; thermoplastic rubber; and combinations of these.

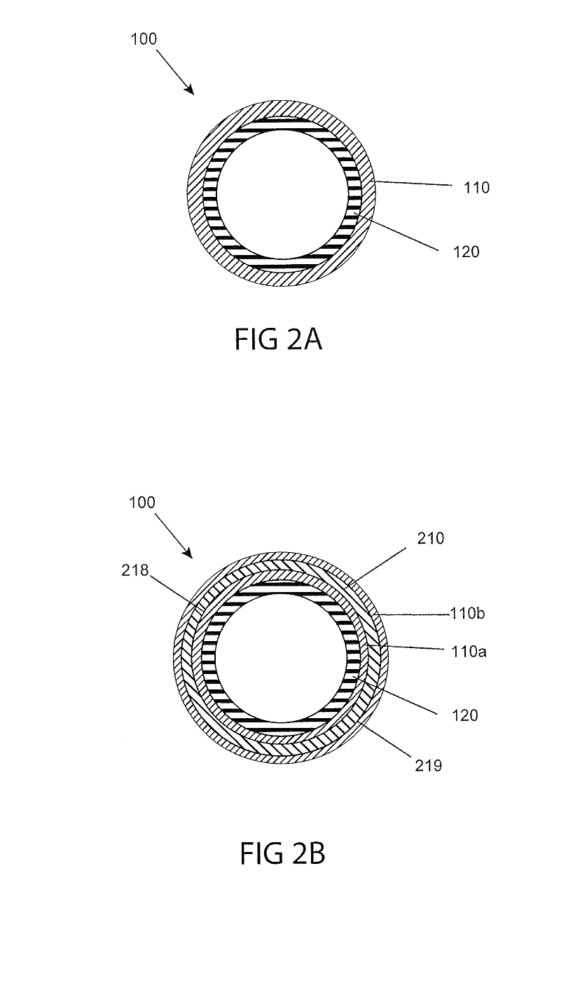

Fiber matrix 110 can comprise one or more layers, such as a fiber matrix 110 with a thickness between 100 μm and 1000 μm, such as a thickness between 150 μm and 400 μm, between 220 μm and 280 μm, or approximately 250 μm. In some embodiments, fiber matrix 110 comprises an inner layer and an outer layer, such as an inner and outer layer with a spine 210 positioned therebetween, as described in reference to FIG. 2B herebelow. Fiber matrix 110 can comprise a matrix of fibers with a width, such as an average diameter (hereinafter “diameter”), of at least 5 μm, such as a diameter between 6 μm and 15 μm, such as a matrix of fibers with an average diameter of approximately 7.8 μm or approximately 8.6 μm. Fiber matrix 110 can comprise an average porosity (hereinafter “porosity”) of between 40% and 80%, such as a fiber matrix with an average porosity of 50.4% or 46.9%. The porosity of fiber matrix 110 can be selected to control infiltration of materials into fiber matrix 110 and/or to control the rate of transmural cellular infiltration within the fiber matrix 110. In some embodiments, fiber matrix 110 comprises an average compliance (hereinafter “compliance”) between approximately 0.2×10⁻⁴/mmHg and 3.0×10⁻⁴/mmHg when measured in arterial pressure ranges. In some embodiments, fiber matrix 110 comprises an average circumferential elastic modulus (hereinafter “elastic modulus”) between 10 MPa and 18 MPa.

Fiber matrix 110 can comprise at least one polymer such as a polymer selected from the group consisting of: polyolefins; polyurethanes; polyvinylchlorides; polyamides; polyimides; polyacrylates; polyphenolics; polystyrene; polycaprolactone; polylactic acid; polyglycolic acid; and combinations of these. The polymer can be applied in combination with a solvent where the solvent is selected from the group consisting of: hexafluoroisopropanol (HFIP); acetone; methyl ethyl ketone; benzene; toluene; xylene; dimethyleformamide; dimethylacetamide; propanol;

ethanol; methanol; propylene glycol; ethylene glycol; trichloroethane; trichloroethylene; carbon tetrachloride; tetrahydrofuran; cyclohexone; cyclohexpropylene glycol; DMSO; tetrahydrofuran; chloroform; methylene chloride; and combinations of these. Fiber matrix 110 can comprise a thermoplastic co-polymer including two or more materials, such as a first material and a harder second material. In some embodiments, the softer material comprises segments including polydimethylsiloxane and polyhexamethylene oxide, and the harder material comprises segments including aromatic methylene diphenyl isocyanate. In some embodiments, fiber matrix 110 comprises relatively equal amounts of the softer and harder materials. In some embodiments, fiber matrix 110 comprises Elast-Eon™ material manufactured by Aortech Biomaterials of Scoresby, Australia, such as model number E2-852 with a durometer of 55 D.

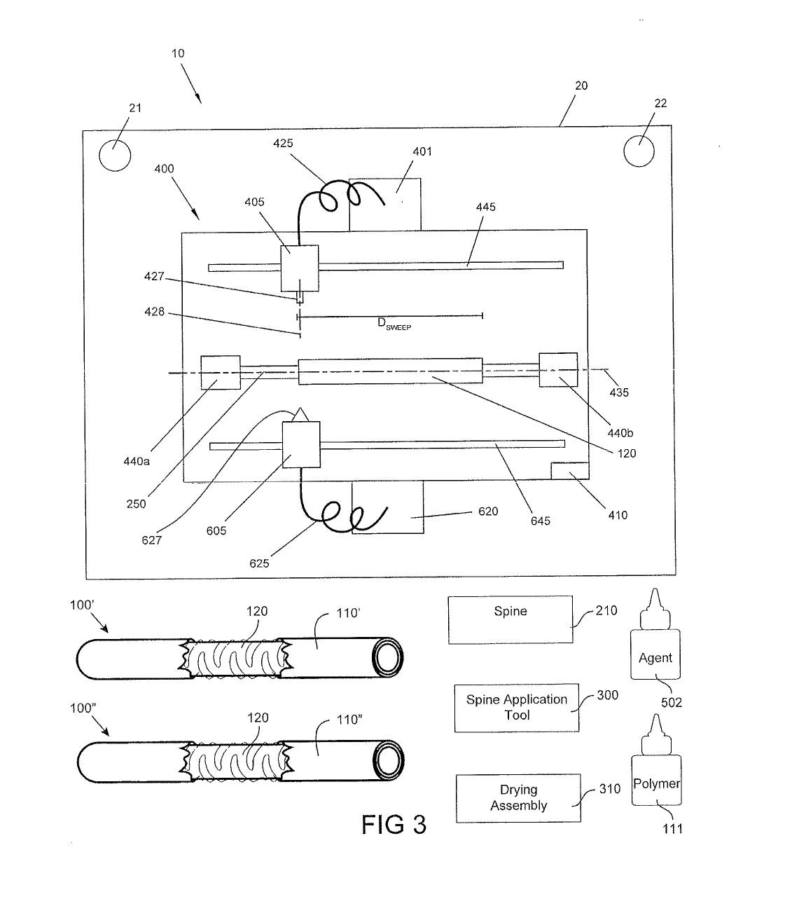

In some embodiments, fiber matrix 110 is produced by a fiber matrix delivery assembly such as an electrospinning device that converts a polymer solution into fibers applied to tubular conduit 120, as described in reference to system 10 and electrospinning device 400 of FIG. 3 described herebelow. The polymer solution can comprise one or more polymers dissolved in a solvent such as hexafluoroisopropanol (HFIP). In some embodiments, at least a portion of fiber matrix 110 is applied with a device selected from the group consisting of: an electrospinning device; a melt-spinning device; a melt-electrospinning device; a misting assembly; a sprayer; an electrosprayer; a three-dimensional printer; and combinations of these.

Fiber matrix 110 can comprise one or more relatively durable (i.e. non-biodegradable) materials and/or one or more biodegradable materials. In some embodiments, fiber matrix 110 comprises a material selected from the group consisting of: polyglycerol sebacate; hyaluric acid; silk fibroin collagen; elastin; poly(p-dioxanone); poly(3-hydroxybutyrate); poly(3-hydroxyvalerate); poly(valcrolactone); poly(tartronic acid); poly(beta-malonic acid); poly(propylene fumarates); a polyanhydride; a tyrosine-derived polycarbonate; a polyorthoester; a degradable polyurethane; a polyphosphazene; and combinations of these. Fiber matrix 110 can comprise one or more drugs or other agents, such as one or more agents constructed and arranged to be released over time.

In some embodiments, graft device 100 further includes one or more kink resisting elements, such as spine 210. Spine 210 can be constructed and arranged to prevent graft device 100 from undergoing undesired motion such as kinking or other narrowing, such as narrowing caused during an implantation procedure and/or while under stresses endured during its functional lifespan. In some embodiments, spine 210 surrounds conduit 120, positioned between conduit 120 and fiber matrix 110. In these embodiments, spine 210 can comprise a diameter approximating the outer diameter (OD) of conduit 120. In some embodiments, spine 210, in whole or in part, can be positioned between one or more layers of fiber matrix 110, such as is shown in FIG. 2B and described herebelow. In some embodiments, spine 210, in whole or in part, can surround the outer surface of fiber matrix 110. In some embodiments, spine 210 is positioned within conduit 120. In some embodiments, multiple spines 210 can be included, each contacting the outer surface of tubular conduit 120, surrounding the outer surface of fiber matrix 110, and/or positioned between two or more layers of fiber matrix 110.

Fiber matrix 110 and/or spine 210 can be constructed and arranged to provide one or more functions selected from the group consisting of: minimizing undesirable conditions, such as buckling or kinking, conduit 120 deformation, luminal deformation, stasis, flows characterized by significant secondary components of velocity vectors such as vortical, recirculating or turbulent flows, luminal collapse, and/or thrombus formation; preserving laminar flow such as preserving laminar flow with minimal secondary components of velocity, such as blood flow through graft device 100, blood flow proximal to graft device 100 and/or blood flow distal to graft device 100; preventing bending and/or allowing proper bending of the graft device 100, such as bending that occurs during and/or after the implantation procedure; preventing accumulation of debris; preventing stress concentration on the tubular wall; maintaining a defined geometry in tubular conduit 120,: preventing axial rotation about the length of tubular conduit 120; and combinations of these. Spine 210 and fiber matrix 110 can comprise similar elastic moduli, such as to avoid dislocations and/or separations between the two components over time, such as when graft device 100 undergoes cyclic motion and/or strain.

Spine 210 can be applied around conduit 120 prior to, during and/or after application of fiber matrix 110 to graft device 100. For example, spine 210 can be applied prior to application of fiber matrix 110 when spine 210 is positioned between conduit 120 and the inner surface of fiber matrix 110. Spine 210 can be applied during application of fiber matrix 110 when spine 210 is positioned between one or more layers of fiber matrix 110, as shown in FIG. 2B. Spine 210 can be applied after application of fiber matrix 110 when spine 210 is positioned outside of fiber matrix 110. Spine 210 can be applied about conduit 120 and/or at least a layer of fiber matrix 110 with one or more tools, such as tool 300 described herebelow in reference to FIG. 3.

Spine 210 can include one or more portions that are resiliently biased, such as a resilient bias configured to provide a radial outward force at locations proximate ends 101 and/or 102, such as to provide a radial outward force to support or enhance the creation of an anastomosis during a cardiovascular bypass procedure. In some embodiments, spine 210 includes one or more portions that are malleable.

Spine 210 can include multiple curved projections 211′ and 211″, collectively 211. Projections 211′ each include a tip portion 212′ and projections 211″ each include a tip portion 212″ (collectively, tip portions 212). Tip portions 212 can be arranged in the overlapping arrangement shown in FIG. 1. Projections 211′ and 211″ can comprise a first and second support portion, respectively, that are arranged such that at least one rotates relative to the other to create an opening to receive tubular conduit 120. In some embodiments, each tip portion 212 can comprise a diameter between 0.020 inches and 0.064 inches, such as a diameter approximating 0.042 inches. Projections 211 can each comprise a loop of a filament (e.g. a loop of a continuous filament), and projections 211′ and 211″ can be arranged in an interdigitating arrangement such as the alternating, interdigitating arrangement shown in FIG. 1. In some embodiments, the interdigitating projections 211′ and 211″ can overlap (e.g. spine 210 covers more than 360° of conduit 120). In some embodiments, projections 211′ and 211″ are arranged with an overlap of at least 1.0 mm, at least 1.1 mm or at least 1.4 mm. In some embodiments, spine 210 can be constructed and arranged as described in applicant's co-pending International Patent Application Ser. No. PCT/US2014/056371, filed Sep. 18, 2014, the contents of which are incorporated herein by reference in their entirety.

Spine 210 can comprise at least three projections 211, such as at least six projections 211. In some embodiments, spine 210 includes at least two projections 211 for every 15 mm of length of spine 210, such as at least two projections 211 for every 7.5 mm of length of spine 210, or at least two projections for every 2 mm of length of spine 210. In some embodiments, spine 210 comprises two projections 211 for each approximately 6.5 mm of length of spine 210. In some embodiments, a series of projections 211 are positioned approximately 0.125 inches from each other.

Spine 210 can comprise one or more continuous filaments 216, such as three or less continuous filaments, two or less continuous filaments, or a single continuous filament. In some embodiments, spine 210 comprises a continuous filament 216 of at least 15 inches long, or at least 30 inches long such as when spine 210 comprises a length of approximately 3.5 inches. In some embodiments, filament 216 comprises a length (e.g. a continuous length or a sum of segments with a cumulative length) of approximately 65 inches (e.g. to create a 4.0 mm diameter spine 210), or a length of approximately 75 inches (e.g. to create a 4.7 mm diameter spine 210), or a length of approximately 85 inches (e.g. to create a 5.5 mm diameter and/or 3.5 inches long spine 210). Filament 216 can comprise a relatively continuous cross section, such as an extruded or molded filament with a relatively continuous cross section. Spine 210 can comprise a filament 216 including at least a portion with a cross sectional geometry selected from the group consisting of: elliptical; circular; oval; square; rectangular; trapezoidal; parallelogram-shaped; rhomboid-shaped; T-shaped; star-shaped; spiral-shaped; (e.g. a filament comprising a rolled sheet); and combinations of these. Filament 216 can comprise a cross section with a major axis between approximately 0.2 mm and 1.5 mm in length, such as a circle or oval with a major axis less than or equal to 1.5 mm, less than or equal to 0.8 mm, or less than or equal to 0.6 mm, or between 0.4 mm and 0.5 mm. Filament 216 can comprise a cross section with a major axis greater than or equal to 0.1 mm, such as a major axis greater than or equal to 0.3 mm. In some embodiments, the major axis and/or cross sectional area of filament 216 is proportionally based to the diameter of spine 210 (e.g. a larger spine 210 diameter correlates to a larger filament 216 diameter, such as when a range of different diameter spine 210′s are provided in a kit as described herebelow in reference to FIG. 3.

Filament 216 can be a single core, monofilament structure. Alternatively, filament 216 can comprise multiple filaments, such as a braided multiple filament structure. In some embodiments, filament 216 can comprise an injection molded component or a thermoset plastic component, such as when spine 210 comprises multiple projections 211 that are created at the same time as the creation of one or more filaments 216 (e.g. when filament 216 is created in a three dimensional biased shape).

Filament 216 can comprise an electrospun component, such as a component fabricated by the same electrospinning device used to create fiber matrix 110, such as when spine 210 and fiber matrix 110 comprise the same or similar materials.

Spine 210 can comprise a material with a durometer between 52 D and 120 R, such as between 52 D and 85 D, such as between 52 D and 62 D. In some embodiments, spine 210 comprises a material with a durometer of approximately 55 D. Spine 210 can comprise one or more polymers, such as a polymer selected from the group consisting of: silicone; polyether block amide; polypropylene; nylon; polytetrafluoroethylene; polyethylene; ultra high molecular weight polyethylene; polycarbonates; polyolefins; polyurethanes; polyvinylchlorides; polyamides; polyim ides; polyacrylates; polyphenolics; polystyrene; polycaprolactone; polylactic acid; polyglycolic acid; polyglycerol sebacate; hyaluric acid; silk fibroin collagen; elastin; poly(p-dioxanone); poly(3-hydroxybutyrate); poly(3-hydroxyvalerate); poly(valcrolactone); poly(tartronic acid); poly(beta-malonic acid); poly(propylene fumarates); a polyanhydride; a tyrosine-derived polycarbonate; a polyorthoester; a degradable polyurethane; a polyphosphazene; and combinations of these.

Spine 210 can comprise the same material as fiber matrix 110. Spine 210 can comprise at least one thermoplastic co-polymer. Spine 210 can comprise two or more materials, such as a first material and a second material harder than the first material. In some embodiments, Spine 210 can comprise relatively equal amounts of a harder material and a softer material. The softer material can comprise polydimethylsiloxane and a polyether-based polyurethane and the harder material can comprise aromatic methylene diphenyl isocyanate. Spine 210 can comprise one or more drugs or other agents, such as one or more agents constructed and arranged to be released over time.

In some embodiments, spine 210 comprises a metal material, such as a metal selected from the group consisting of: nickel titanium alloy; titanium alloy; titanium; stainless steel; tantalum; magnesium; cobalt-chromium alloy; gold; platinum; and combinations of these. In some embodiments, spine 210 comprises a reinforced resin, such as a resin reinforced with carbon fiber and/or Kevlar. In some embodiments, at least a portion of spine 210 is biodegradable, such as when spine 210 comprises a biodegradable material such as a biodegradable metal or biodegradable polymer. In these embodiments, fiber matrix 110 can comprise a biodegradable material and/or a non-biodegradable material. In some embodiments, spine 210 does not comprise a biodegradable material. In these embodiments, fiber matrix 110 can comprise a biodegradable material and/or a non-biodegradable material.

Spine 210 can be configured to biodegrade over time such as to provide a temporary kink resistance or other function to device 100. In one embodiment, spine 210 can temporarily provide kink resistance to graft device 100 for a period of less than twenty-four hours. In an alternative embodiment, spine 210 can provide kink resistance to graft device 100 for a period of less than one month. In yet another embodiment, spine 210 can provide kink resistance to graft device 100 for a period of less than six months. Numerous forms of biodegradable materials can be employed. Bolz et al. (U.S. patent Ser. No. 09/339,927) discloses a bioabsorbable implant which includes a combination of metal materials that can be an alloy or a local galvanic element. Metal alloys can consist of at least a first component which forms a protecting passivation coat and a second component configured to ensure sufficient corrosion of the alloy. The first component is at least one component selected from the group consisting of: magnesium, titanium, zirconium, niobium, tantalum, zinc and silicon, and the second component is at least one metal selected from the group consisting of: lithium, sodium, potassium, manganese, calcium and iron. Furst et al. (U.S. patent application Ser. No. 11/368,298) discloses an implantable device at least partially formed of a bioabsorbable metal alloy that includes a majority weight percent of magnesium and at least one metal selected from calcium, a rare earth metal, yttrium, zinc and/or zirconium. Doty et al. (U.S. patent application Ser. No. 11/744,977) discloses a bioabsorbable magnesium reinforced polymer stent that includes magnesium or magnesium alloys. Numerous biodegradable polymers can be used such as are described hereabove.

Fiber matrix 110 and/or spine 210 can comprise one or more coatings. The one or more coatings can comprise an adhesive element or otherwise exhibit adhesive properties, such as a coating comprising a material selected from the group consisting of: fibrin gel; a starch-based compound; mussel adhesive protein; and combinations of these. The coating can be constructed and arranged to provide a function selected from the group consisting of: anti-thrombogenecity; anti-proliferation; anti-calcification; vasorelaxation; and combinations of these. A coating can comprise a dehydrated gelatin, such as a dehydrated gelatin coating configured to hydrate to cause adherence of two or more of tubular conduit 120, fiber matrix 110 and spine 210. A coating can comprise a hydrophilic and/or a hydrophobic coating. A coating can comprise a radiopaque coating. In some embodiments, spine 210 comprises at least a portion that is radiopaque, such as when spine 210 comprises a radiopaque material such as barium sulfate.

In some embodiments, device 100 is constructed and arranged to be placed in an in-vivo geometry including one or more arced portions including a radius of curvature of as low as 0.5 cm (e.g. without kinking). In some embodiments, device 100 is produced using system 10 and/or electrospinning device 400 of FIG. 3, as described herebelow.

Referring now to FIG. 2A, a sectional view of one embodiment of the graft device of FIG. 1 is illustrated, comprising a tubular conduit and a surrounding fiber matrix, consistent with the present inventive concepts. Graft device 100 includes tubular conduit 120. A fiber matrix 110 has been applied about the surface of conduit 120, such as is described in detail herebelow in reference to FIG. 3. Fiber matrix 110 can comprise one or more polymers, such as a combination of polydimethylsiloxane and polyhexamethylene oxide soft segments, and aromatic methylene diphenyl isocyanate hard segments. Fiber matrix 110 can comprise a thickness of between 220 μm and 280 μm, such as a thickness of approximately 250 μm.

Referring now to FIG. 2B, a sectional view of another embodiment of the graft device of FIG. 1 is illustrated, including a spine placed between layers of a fiber matrix, consistent with the present inventive concepts. In the embodiment of FIG. 2B, spine 210 has been placed between one or more inner layers of fiber, inner layer 110a, and one or more outer layers of fiber, outer layer 110b. In these embodiments, spine 210 can be applied (e.g. laterally applied) to conduit 120 after inner layer 110a has been applied to conduit 120 by an electrospinning device or other fiber matrix delivery assembly, as described herein, such as by interrupting the delivery of fiber to conduit 120, to apply spine 210 over the already applied inner layer 110a. In some embodiments, inner layer 110a comprises a thickness approximately one-half the thickness of outer layer 110b. In some embodiments, inner layer 110a comprises a thickness of approximately between 62 μm and 83 μm. In some embodiments, inner layer 110a comprises between 1% and 99% of the total thickness of fiber matrix 110, such as between 25% and 60% of the total thickness, or approximately 33% of the total thickness of fiber matrix 110. In some embodiments, the process time of applying inner layer 110a is between 1% and 99% of the total application time (i.e. the collective time to apply inner layer 110a and outer layer 110b), such as between 25% and 60% of the total fiber application time, or approximately 33% of the total fiber application time.

Spine 210 comprises an inner surface 218 which contacts the outer surface of inner layer 110a. Spine 210 further comprises an outer surface 219 which contacts the inner surface of outer layer 110b. Inner surface 218, outer surface 219 and/or another surface of spine 210 can comprise a coating, such as a coating described hereabove.

Application of layers 110a and 110b can be performed as is described in detail herebelow in reference to FIG. 3. Fiber matrix layers 110a and/or 110b can comprise one or more polymers, such as a combination of polydimethylsiloxane and polyhexamethylene oxide soft segments, and aromatic methylene diphenyl isocyanate hard segments. Layers 110a and/or 110b can comprise a matrix of fibers with a diameter between 6 μm and 15 μm, such as a matrix of fibers with an average diameter of approximately 7.8 μm or approximately 8.6 μm. Layers 110a and/or 110b can comprise a porosity of between 40% and 80%, such as a fiber matrix with an average porosity of 50.4% or 46.9%. In some embodiments, layers 110a and/or 110b comprise a compliance between approximately 0.2×10⁻⁴/mmHg and 3.0×10⁻⁴/mmHg when measured in arterial pressure ranges. In some embodiments, fiber matrix 110 comprises an elastic modulus between 10 MPa and 18 MPa.

Referring now to FIG. 3, a schematic view of a system for producing a graft device with an electrospun fiber matrix is illustrated, consistent with the present inventive concepts. System 10 includes a fiber matrix delivery assembly, electrospinning device 400. System 10 is constructed and arranged to produce one or more graft devices, such as graft device 100′ or 100″ shown (collectively graft device 100), each including a fiber matrix, such as fiber matrix 110′ or 110″, respectively (collectively fiber matrix 110), the fiber matrix 110 surrounding a tubular conduit 120. System 10 includes mandrel 250 about which a tubular conduit 120 has been placed. System 10 can include polymer material 111, including a mixture of one or more polymers, solvents and/or other materials used to create fiber matrix 110, such as are described hereabove in reference to FIG. 1. Tubular conduit 120 can include living tissue and/or artificial materials. In some embodiments, system 10 comprises one or more similar or dissimilar spines 210, and graft device 100 comprises one or more of the spines 210. System 10 can include spine application tool 300, which can comprise a manual or automated (e.g. robotic) tool used to place spine 210 about tubular conduit 120, such as between one or more layers of fiber matrix 110 (e.g. between an inner layer with a first thickness, and an outer layer with a second thickness approximately twice as thick as the first layer's thickness). In some embodiments, device 100, fiber matrix 110, spine 210 and/or conduit 120 are as is described hereabove in reference to FIG. 1. In some embodiments, system 10 can include one or more tools, components, assemblies and/or otherwise be constructed and arranged as described in applicant's co-pending International Patent Application Serial Number PCT/U.S. 2014/056371, filed Sep. 18, 2014, the contents of which are incorporated herein by reference in their entirety.

Mandrel 250 can comprise a metal mandrel, such as a mandrel constructed of 304 or 316 series stainless steel. Mandrel 250 can comprise a mirror-like surface finish, such as a surface finish with an Rₐ of approximately 0.1 μm to 0.8 μm. Mandrel 250 can comprise a length of up to 45 cm, such as a length of between 30 cm and 45 cm, or between 38 cm and 40 cm. In some embodiments, system 10 includes multiple mandrels 250 with multiple different geometries, such as a set of mandrels 250 with different diameters (e.g. diameters of 3.0 mm, 3.5 mm, 4.0 mm and/or 4.5 mm). Each end of mandrel 250 is inserted into a rotating assembly, motors 440a and 440b, respectively, such that mandrel 250 can be rotated about axis 435 during application of fiber matrix 110. In some embodiments, a single motor drives one end of mandrel 250, with the opposite end attached to a rotatable attachment element of electrospinning device 400.

Electrospinning device 400 can include one or more polymer delivery assemblies, and in the illustrated embodiment, device 400 includes polymer delivery assembly 405, which includes nozzle 427 including an orifice constructed and arranged to deliver fiber matrix 110 to tubular conduit 120. Nozzle 427 can be a tubular structure including nozzle central axis 428. Polymer delivery assembly 405 is fluidly attached to polymer solution dispenser 401 via delivery tube 425. Polymer solution dispenser 401 can comprise material supplied by polymer material 111 (e.g. when polymer material 111 comprises one or more polymers contained in a cartridge that is operably received by polymer solution dispenser 401). Polymer delivery assembly 405 is operably attached to a linear drive assembly 445 configured to translate polymer delivery assembly 405 in at least one direction for a linear travel distance D_{SWEEP} as shown. In some embodiments, D_{SWEEP} comprises a length of approximately 30 cm, such as a length of at least 10 cm, 20 cm, 30 cm, 35 cm or 40 cm.

In some embodiments, polymer material 111 comprises two or more polymers, such as a first polymer with a first hardness, and a second polymer with a second hardness different than the first hardness. Polymer material can comprise a mixture of similar or dissimilar amounts of polyhexamethylene oxide soft segments, and aromatic methylene diphenyl isocyanate hard segments. Polymer material 111 can further comprise one or more solvents, such as HFIP (e.g. HFIP with a 99.97% minimum purity). Polymer material 111 can comprise one or more polymers in a concentrated solution fully or at least partially solubilized within a solvent and comprise a polymer weight to solvent volume ratio between 20% and 35%, a typical concentration is between 24% and 26% (more specifically between 24.5% and 25.5%). Polymer material 111 can comprise one or more materials with a molecular weight average (M_{w}) between 80,000 and 150,000 (PDIM−M_{w}/Mₙ=2.1-3.5). Polymer material 111 can comprise a polymer solution with a viscosity between 2000 cP and 2400 cP (measured at 25° C. and with shear rate=20 s⁻¹). Polymer material 111 can comprise a polymer solution with a conductivity between 0.4 μS/cm and 1.70/cm (measured at a temperature between 20° C. and 22° C.). Polymer material 111 can comprise a polymer solution with a surface tension between 21.5 mN/m and 23.0 mN/m (measured at 25° C.).

In some embodiments, system 10 is constructed and arranged to produce a fiber matrix 110 with a thickness (absent of any spine 210) of between approximately 220 μm and 280 μm. Fiber matrix 110 can comprise a matrix of fibers with a diameter between 6 μm and 15 μm, such as a matrix of fibers with an average diameter of approximately 7.8 μm or approximately 8.6 μm. Fiber matrix 110 can comprise a porosity of between 40% and 80%, such as a fiber matrix with an average porosity of 50.4% or 46.9%. In some embodiments, fiber matrix 110 comprises a compliance between approximately 0.2×10⁻⁴/mmHg and 3.0×1.0⁴/mmHg when measured in arterial pressure ranges. In some embodiments, fiber matrix 110 comprises an elastic modulus between 10 MPa and 18 MPa.

Polymer delivery assembly 405 can be configured to deliver polymer material 111 to nozzle 427 at a flow rate of between 10 ml/hr and 25 ml /hr, such as at a flow rate of approximately 15 ml/hr or 20 ml/hr.

As described above, in some embodiments, system 10 is constructed and arranged to produce a graft device 100 including a spine 210. Spine 210 can comprise multiple spines 210 with different inner diameters (IDs), such as multiple spines with IDs of approximately 4.0 mm, 4.7 mm and/or 5.5 mm. Spine 210 can comprise a filament with a diameter of approximately 0.4 mm (e.g. for a spine with an ID between 4.0 mm and 4.7 mm). Spine 210 can comprise a filament with a diameter of approximately 0.5 mm (e.g. for a spine with an ID between 4.8 mm and 5.5 mm). Spine 210 can comprise a series of interdigitating fingers spaced approximately 0.125 inches from each other so that the recurring unit of spine including one left finger and one right finger occurs every 0.25 inches. This recurring feature length can have a range comprised between 0.125 inches and 0.375 inches. The fingers can overlap in a symmetric or asymmetric pattern, such as an overlap of opposing fingers between 2.5 mm and 1.0 mm around the circumferential perimeter of spine 210. Spine 210 can be heat treated to achieve a resilient bias. Spine 210 can be surface-treated (e.g. with dimethylformamide) to increase the surface roughness and reduce crystallinity (e.g. to improve solvent-based adhesion with the deposited electrospun material, fiber matrix 110).

System 10 can include a drying assembly 310 constructed and arranged to remove moisture from tubular conduit 120. In some embodiments, tubular conduit 120 comprises harvested tissue (e.g. a harvested saphenous vein segment) and drying assembly 310 comprises gauze or other material used to manually remove fluids from tubular conduit 120, such as to improve adherence between fiber matrix 110 and tubular conduit 120.

Electrospinning device 400 can include one or more graft modification assemblies constructed and arranged to modify one or more components and/or one or more portions of graft device 100. In the illustrated embodiment, device 400 includes modification assembly 605, which includes modifying element 627. Modification assembly 605 is operably attached to a linear drive assembly 645 configured to translate modification assembly 605 in at least one direction, such as a reciprocating motion in back and forth directions spanning a distance similar to D_{SWEEP} of linear drive assembly 445. Modification assembly 605 can be operably attached to supply 620 via delivery tube 625. System 10 can include one or more graft device 100 modifying agents, such as agent 502. Agent 502 can comprise a solvent configured to perform a surface modification, such as a solvent selected from the group consisting of: dimethylformamide; hexafluoroisopropanol; tetrahydrofuran; dimethyl sulfoxide; isopropyl alchohol; ethanol; and combinations of these. In some embodiments, system 10 is constructed and arranged to perform a surface modification configured to enhance the adhesion of two or more of tubular conduit 120, spine 210 and fiber matrix 110. In some embodiments, system 10 is constructed and arranged to perform a surface modification to fiber matrix 110 and/or spine 210 to cause a modification of the surface energy of fiber matrix 110 and/or spine 210, respectively. In some embodiments, the surface of spine 210 is modified with a heated die comprising a textured or otherwise non-uniform surface. In some embodiments, electrospinning device 400 and/or another component of system 10 comprise a radiofrequency plasma glow discharge assembly constructed and arranged to perform a surface modification of spine 210, such as a process performed in the presence of a material selected from the group consisting of: hydrogen; nitrogen; ammonia; oxygen; carbon dioxide; C2F6; C2F4; C3F6; C2H4; CZHZ; CH4; and combinations of these

Supply 620 can comprise one or more of: a reservoir of one or more agents such as agent 502; a power supply such as a laser power supply; and a reservoir of compressed fluid. In some embodiments, modifying element 627 comprises a nozzle, such as a nozzle configured to deliver a fiber matrix 110 modifying agent, tubular conduit 120 modifying agent, spine 210 modifying agent, and/or a graft 100 modifying agent. For clarification, any reference to a “nozzle” or “assembly”, in singular or plural form, can include one or more nozzles, such as one or more nozzles 427, or one or more assemblies, such as one or more polymer delivery assemblies 405 or one or more modification assemblies 605.

In some embodiments, modifying element 627 is configured to deliver an agent 502 comprising a wax or other protective substance to tubular conduit 120 prior to the application of fiber matrix 110, such as to prevent or otherwise minimize exposure of tubular conduit 120 to one or more solvents (e.g. HFIP) included in polymer material 111.

In some embodiments, modifying element 627 is configured to deliver a kink resisting element, for example spine 210, such as a robotic assembly constructed and arranged to laterally deliver spine 210 about at least conduit 120 (e.g. about conduit 120 and an inner layer of fiber matrix 110). Alternatively or additionally, modifying element 627 can be configured to modify conduit 120, spine 210 and/or fiber matrix 110, such as to cause graft device 100 to be kink resistant or otherwise enhance the performance of the graft device 100 produced by system 10. In these graft device 100 modifying embodiments, modifying element 627 can comprise a component selected from the group consisting of: a robotic device such as a robotic device configured to apply spine 210 to tubular conduit 120; a nozzle, such as a nozzle configured to deliver agent 502; an energy delivery element such as a laser delivery element such as a laser excimer diode or other element configured to trim one or more components of graft device 100; a fluid jet such as a water jet or air jet configured to deliver fluid during the application of fiber matrix 110 to conduit 120; a cutting element such as a cutting element configured to trim spine 210 and/or fiber matrix 110; a mechanical abrader; and combinations of these. Modification of fiber matrix 110 or other graft device 100 component by modifying element 627 can occur during the application of fiber matrix 110 and/or after fiber matrix 110 has been applied to conduit 120. Modification of one or more spines 210 can be performed prior to and/or after spine 210 has been applied to surround conduit 120. In some embodiments, modifying element 627 can be used to cut or otherwise trim fiber matrix 110 and/or a spine 210.

In some embodiments, modification assembly 605 of system 10 can be an additional component, separate from electrospinning device 400, such as a handheld device configured to deliver spine 210. In some embodiments, modification assembly 605 comprises a handheld laser, such as a laser device which can be hand operated by an operator. Modification assembly 605 can be used to modify graft device 100 after removal from electrospinning device 400, such as prior to and/or during an implantation procedure.

Laser or other modifications to fiber matrix 110 can cause portions of fiber matrix 110 to undergo physical changes, such as hardening, softening, melting, stiffening, creating a resilient bias, expanding, and/or contracting, and/or can also cause fiber matrix 110 to undergo chemical changes, such as forming a chemical bond with an adhesive layer between the outer surface of conduit 120 and fiber matrix 110. In some embodiments, modifying element 627 is alternatively or additionally configured to modify tubular conduit 120, such that tubular conduit 120 comprises a kink resisting or other performance enhancing element. Modifications to tubular conduit 120 can include but are not limited to a physical change to one or more portions of tubular conduit 120 selected from the group consisting of: drying; hardening; softening; melting; stiffening; creating a resilient bias; expanding; contracting; and combinations of these. Modifications of tubular conduit 120 can cause tubular conduit 120 to undergo chemical changes, such as forming a chemical bond with an adhesive layer between an outer surface of conduit 120 and spine 210 and/or fiber matrix 110.

As described herein, fiber matrix 110 can include an inner layer and an outer layer, where the inner layer can include an adhesive component and/or exhibit adhesive properties. The inner layer can be delivered separate from the outer layer, for example, delivered from a separate nozzle or at a separate time during the process. Selective adhesion between the inner and outer layers can be configured to provide kink resistance. Spine 210 can be placed between the inner and outer layers of fiber matrix 110, such as is described in reference to FIG. 2B hereabove.

In some embodiments, electrospinning device 400 can be configured to deliver fiber matrix 110 and/or an adhesive layer according to set parameters configured to produce a kink resistant element in and/or provide kink resisting properties to device 100. For example, an adhesive layer can be delivered to conduit 120 for a particular length of time, followed by delivery of a polymer solution for another particular length of time. Other typical application parameters include but are not limited to: amount of adhesive layer and/or polymer solution delivered; rate of adhesive layer and/or polymer solution delivered; nozzle 427 distance to mandrel 250 and/or conduit 120; linear travel distance of nozzle 427 or a fiber modifying element along its respective drive assembly (for example, drive assembly 445 or 645); linear travel speed of nozzle 427 or a fiber modifying element along its respective drive assembly; compositions of the polymer solution and/or adhesive layer; concentrations of the polymer solution and/or adhesive layer; solvent compositions and/or concentrations; fiber matrix 110 inner and outer layer compositions and/or concentrations; spontaneous or sequential delivery of the polymer solution and the adhesive layer; voltage applied to the nozzle; voltage applied to the mandrel; viscosity of the polymer solution; temperature of the treatment environment; relative humidity of the treatment environment; airflow within the treatment environment; and combinations of these.

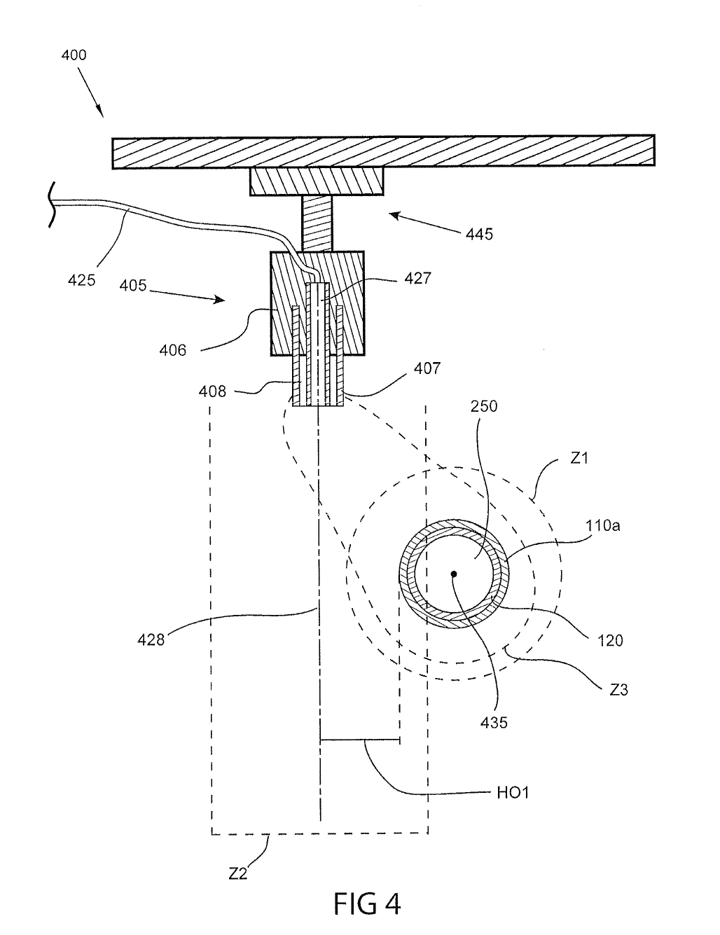

Nozzle 427 can be constructed of stainless steel, such as passivated 304 stainless steel. In some embodiments, nozzle 427 and polymer delivery assembly 405 are constructed and arranged as described herebelow in reference to FIG. 4. A volume of space surrounding nozzle 427 can be maintained free of objects or substances which can interfere with the electrospinning process, also as described herebelow in reference to FIG. 4. In some embodiments, this “object-free” space is void of any objects that contain a charge and/or are electrically conductive. Alternatively or additionally, the object-free space can be void of any objects that may be in a location that would interfere with (e.g. collide with) the flight path of a fiber or any other material traveling between a nozzle (e.g. nozzle 427 or 627) and the tubular conduit 120 and/or fiber matrix 110. Nozzle geometry and orientation, as well as the electrical potential voltages applied between nozzle 427 and mandrel 250 are chosen to control fiber generation, such as to create a fiber matrix 110 as described in reference to FIG. 1 hereabove.

Mandrel 250 is positioned in a particular spaced relationship from polymer delivery assembly 405 and/or modification assembly 605, and nozzle 427 and/or modifying element 627, respectively. In the illustrated embodiment, mandrel 250 is positioned above and below assemblies 605 and 405, respectively. Alternatively, mandrel 250 can be positioned either above, below, to the right and/or or to the left of, assembly 405 and/or assembly 605. The distance between mandrel 250 and the tip of nozzle 427 and/or modifying element 627 can be less than 20 cm, or less than 15 cm, such as distance of between 12.2 cm and 12.8 cm or approximately 12.5 cm. In some embodiments, multiple nozzles 427 and/or multiple modifying elements 627, for example components of similar or dissimilar configurations, can be positioned in various orientations relative to mandrel 250. In some embodiments, the distance between nozzles 427 and/or modifying elements 627 and mandrel 250 varies along the length of their respective travel along mandrel 250, such as to create a varying pattern of fiber matrix 110 along conduit 120. In this embodiment, nozzle 427 and/or modifying element 627 distances from mandrel 250 can vary continuously during the electrospinning process and/or the distance can vary for one or more set periods of time during the process.

In a typical embodiment, an electrical potential is applied between nozzle 427 and one or both of conduit 120 and mandrel 250. The electrical potential can draw at least one fiber from polymer delivery assembly 405 to conduit 120. Conduit 120 can act as the substrate for the electrospinning process, collecting the fibers that are drawn from polymer delivery assembly 405 by the electrical potential. In some embodiments, mandrel 250 and/or conduit 120 has a lower voltage than nozzle 427 to create the desired electrical potential. For example, the voltage of mandrel 250 and/or conduit 120 can be a negative or zero voltage while the voltage of nozzle 427 can be a positive voltage. Mandrel 250 and/or conduit 120 can have a voltage of about −5 kV (e.g., −10 kV, −9 kV, −8 kV, −7 kV, −6 kV, −5 kV, −4.5 kV, −4 kV, −3.5 kV, −3.0 kV, −2.5 kV, −2 kV, −1.5 kV or −1 kV) and the nozzle 427 can have a voltage of about +15 kV (e.g., 2.5 kV, 5 kV, 7.5 kV, 12 kV, 13.5 kV, 15 kV, 17 kV or 20 kV). In some embodiments, the potential difference between nozzle 427 and mandrel 250 and/or conduit 120 can be from about 5 kV to about 30 kV. This potential difference draws fibers from nozzle 427 to conduit 120. In some embodiments, nozzle 427 is electrically charged with a potential of between +15 kV and +17 kV while mandrel 250 is at a potential of approximately −2kV. In some embodiments, mandrel 250 is a fluid mandrel, such as the fluid mandrel described in applicant's co-pending PCT Application Serial Number PCT/U.S. 2011/066905 filed on Dec. 22, 2011, the contents of which are incorporated herein by reference in their entirety.

In some embodiments, system 10 comprises a polymer solution, such as polymer material 111. Polymer material 111 can be introduced into polymer solution dispenser 401, and then delivered to polymer delivery assembly 405 through polymer solution delivery tube 425. The electrical potential between nozzle 427 and conduit 120 and/or mandrel 250 can draw the polymer solution through nozzle 427 of polymer delivery assembly 405. Electrostatic repulsion, caused by the fluid becoming charged from the electrical potential, counteracts the surface tension of a stream of the polymer solution at nozzle 427 of the polymer delivery assembly 405. After the stream of polymer solution is stretched to its critical point, one or more streams of polymer solution emerges from nozzle 427 of polymer delivery assembly 405, and/or at a location below polymer delivery assembly 405, and move toward the negatively charged conduit 120. Using a volatile solvent, the solution dries substantially during transit and fiber is applied about conduit 120 creating fiber matrix 110.

Mandrel 250 is configured to rotate about an axis, such as central axis 435 of mandrel 250, with axis 428 of nozzle 427 typically oriented orthogonal to axis 435. In some embodiments, axis 428 of nozzle 427 is horizontally offset from axis 435, such as is described herebelow in reference to FIG. 4. The rotation around axis 435 allows fiber matrix 110 to be applied along all sides, or around the entire circumference of conduit 120. In some embodiments, two motors 440a and 440b are used to rotate mandrel 250. Alternatively, electrospinning device 400 can include a single motor configured to rotate mandrel 250 as described hereabove. The rate of rotation of mandrel 250 can determine how the electrospun fibers are applied to one or more segments of conduit 120. For example, for a thicker portion of fiber matrix 110, the rotation rate can be slower than when a thinner portion of fiber matrix 110 is desired. In some embodiments, mandrel 250 is rotated at a rate of between 100 rpm and 400 rpm, such as a rate of between 200 rpm and 300 rpm, between 240 rpm and 260 rpm, or approximately 250 rpm.

In addition to mandrel 250 rotating around axis 435, the polymer delivery assembly 405 can move, such as when driven by drive assembly 445 in a reciprocating or oscillating horizontal motion (to the left and right of the page). Drive assembly 445, as well as drive assembly 645 which operably attaches to modification assembly 605, can each comprise a linear drive assembly, such as a belt-driven drive assembly comprising two or more pulleys driven by one or more stepper motors. Additionally or alternatively, assemblies 405 and/or 605 can be constructed and arranged to rotate around axis 435, rotating means not shown. The length of drive assemblies 445 and/or 645 and the linear motion applied to assemblies 405 and 605, respectively, can vary based on the length of conduit 120 to which a fiber matrix 110 is delivered and/or a fiber matrix 110 modification is applied. For example, the supported linear motion of drive assemblies 445 and/or 645 can be about 10 cm to about 50 cm, such as to cause a translation of assembly 405 and/or 605 between 27 cm and 31 cm, or approximately 29 cm. Rotational speeds of mandrel 250 and translational speeds of assemblies 405 and/or 605 can be relatively constant, or can be varied during the fiber application process. In some embodiments, assembly 405 and/or 605 are translated (e.g. back and forth) at a relatively constant translation rate between 40 mm/sec and 150 mm/sec, such as to cause nozzle 427 and/or modifying element 627 to translate at a rate of between 50 mm/sec and 80 mm/sec, between 55 mm/sec and 65 mm/sec, or approximately 60 mm/sec, during the majority of its travel. In some embodiments, system 10 is constructed and arranged to rapidly change directions of translation (i.e. maximize deceleration before a direction change and/or maximize acceleration after a direction change).

Assemblies 405 and/or 605 can move along the entire length or specific portions of the length of conduit 120. In some embodiments, fiber and/or modification is applied to the entire length of conduit 120 plus an additional 5 cm (to mandrel 250) on either or both ends of conduit 120. In another embodiment, fiber(s) and/or modification is applied to the entire length of conduit 120 plus at least 1 cm beyond either or both ends of conduit 120. Assemblies 405 and/or 605 can be controlled such that specific portions along the length of conduit 120 are reinforced with a greater amount of fiber matrix 110 as compared to other or remaining portions. Alternatively or additionally, assemblies 405 and/or 605 can be controlled such that specific portions of the length of conduit 120 include one or more kink resistant elements (e.g. one or more spines 210) positioned at those one or more specific conduit 120 portions. In addition, conduit 120 can be rotating around axis 435 while assemblies 405 and/or 605 move, via drive assemblies 445 and/or 645, respectively, to position assemblies 405 and/or 605 at the particular portion of conduit 120 to which fiber is applied and/or modified.

System 10 can also include a power supply, power supply 410 configured to provide the electric potentials to nozzle 427 and mandrel 250, as well as to supply power to other components of system 10 such as drive assemblies 445 and 645 and modification assembly 605. Power supply 410 can be connected, either directly or indirectly, to at least one of mandrel 250 and conduit 120. Power can be transferred from power supply 410 to each component by, for example, one or more wires.

System 10 can include an environmental control assembly including environmental chamber 20 that surrounds electrospinning device 400. System 10 can be constructed and arranged to control the environmental conditions within chamber 20, such as to control one or more environment surrounding polymer delivery assembly 405 and/or mandrel 250 during the application of fiber matrix 110 to conduit 120. Chamber 20 can include inlet port assembly 21 and outlet port assembly 22. Inlet port assembly 21 and/or outlet port assembly 22 can each include one or more components such as one or more components selected from the group consisting of: a fan; a source of a gas such as a dry compressed air source; a source of gas at a negative pressure; a vapor source such as a source including a buffered vapor, an alkaline vapor and/or an acidic vapor; a filter such as a HEPA filter; a dehumidifier; a humidifier; a heater; a chiller; and electrostatic discharge reducing ion generator; and combinations of these. Chamber 20 can include one or more environmental control components to monitor and/or control temperature, humidity and/or pressure within chamber 20. Chamber 20 can be constructed and arranged to provide relatively uniform ventilation about mandrel 250 (e.g. about tubular conduit 120, fiber matrix 110 and/or spine 210) including an ultra-dry (e.g. ≦2 ppm water or other liquid content) compressed gas (e.g. air) source to reduce humidity. Inlet port 21 and outlet port 22 can be oriented to purge air from the top of chamber 20 to the bottom of chamber 20 (e.g. to remove vapors of one or more solvents (e.g. HFIP) which can tend to settle at the bottom of chamber 20). Chamber 20 can be constructed and arranged to replace the internal volume of chamber 20 at least once every 3 minutes, or once every 1 minutes, or once every 30 seconds. Outlet port 22 can include one or more filters (e.g. replaceable cartridge filters) which are suitable for retaining halogenated solvents or other undesired materials evacuated from chamber 20. Chamber 20 can be constructed and arranged to maintain a flow rate through chamber 20 of at least 30 L/min, such as at least 45 L/min or at least 60 L/min during an initial purge procedure. Subsequent to the initial purge procedure, a flow rate of at least 5 L/min, at least 10 L/min, at least 20 L/min or at least 30 L/min can be maintained, such as to maintain a constant humidity level (e.g. a relative humidity between 20% and 24%). Chamber 20 can be further constructed and arranged to control temperature, such as to control temperature within chamber 20 to a temperature between 15° C. and 25° C., such as between 16° C. and 20° C. with a relative humidity between 20% and 24%. In some embodiments, one or more objects or surfaces within chamber 20 are constructed of an electrically insulating material and/or do not include sharp edges or exposed electrical components. In some embodiments, one or more metal objects positioned within chamber 20 are electrically grounded.

In some embodiments, system 10 is configured to produce a graft device 100′ based on one or more component or process parameters. In these embodiments, graft device 100′ comprises tubular conduit 120 and a fiber matrix 110′ applied by electrospinning device 400. Fiber matrix 110′ can be applied via polymer delivery assembly 405 supplied with polymer material 111 at a flow rate of approximately 15 ml/hr. Fiber matrix 110′ can be applied when an electrostatic potential of approximately 17 kV is applied between nozzle 427 and mandrel 250, such as when nozzle 427 is charged to a potential of approximately +15 kV and mandrel 250 is charged to a potential of approximately −2 kV. Cumulative application time of fiber matrix 110′ can comprise an approximate time period of between 11 minutes and 40 seconds and 17 minutes and 30 seconds. The cumulative application time of fiber matrix 110′ can comprise a time period of approximately 11 minutes and 40 seconds when tubular conduit 120 comprises an outer diameter of between approximately 3.4 mm and 4.2 mm, a time period of approximately 14 minutes and 0 seconds when tubular conduit 120 comprises an outer diameter between approximately 4.2 mm and 5.1 mm, and/or a time period of approximately 17 minutes and 30 seconds when tubular conduit 120 comprises an outer diameter between approximately 5.1 mm and 6.0 mm.

Fiber matrix 110′ can comprise an average fiber size of approximately 7.8 μm, such as a population of fiber diameters with an average fiber size of approximately 7.8 μm with a standard deviation of 0.45 μm. Fiber matrix 110′ can comprise an average porosity of approximately 50.4%, such as a range of porosities with an average of 50.4% and a standard deviation of 1.1%. Fiber matrix 110′ can comprise a strength property selected from the group consisting of: stress measured at 5% strain comprising between 0.4 MPa and 1.1 MPa; ultimate stress of 4.5 MPa to 7.0 MPa; ultimate strain of 200% to 400%; and combinations of these. Fiber matrix 110′ can comprise a compliance between approximately 0.2×10⁴/mmHg and 3.0×10⁻⁴/mmHg when measured in arterial pressure ranges. Fiber matrix 110′ can comprise an elastic modulus between 10 MPa and 15 MPa. Fiber matrix 110′ can be constructed and arranged with a targeted suture retention strength, such as an approximate suture retention strength of between 2.0 N and 4.0 N with 6-0 Prolene suture and/or between 1.5 N and 3.0 N with 7-0 Prolene suture. In some embodiments, graft device 100′ includes a spine 210, such as a spine 210 placed between inner and outer layers of fiber matrix 110′ (e.g. placed after one-third of the total thickness of fiber matrix 110′ is applied about conduit 120).

In some embodiments, system 10 is configured to produce a graft device 100″ based on one or more component or process parameters. In these embodiments, graft device 100″ comprises tubular conduit 120 and a fiber matrix 110″ applied by electrospinning device 400. Fiber matrix 110″ can be applied via polymer delivery assembly 405 supplied with polymer material 111 at a flow rate of approximately 20 ml/hr. Fiber matrix 110″ can be applied when an electrostatic potential of approximately 19 kV is applied between nozzle 427 and mandrel 250, such as when nozzle 427 is charged to a potential of approximately +17kV and mandrel 250 is charged to a potential of approximately −2 kV. Cumulative application time of fiber matrix 110″ can comprise an approximate time period of between 9 minutes and 30 seconds and 13 minutes and 40 seconds. The cumulative application time of fiber matrix 110″ can comprise a time period of approximately 9 minutes and 30 seconds when tubular conduit 120 comprises an outer diameter between approximately 3.4 mm and 4.2 mm; a time period of approximately 11 minutes and 30 seconds when tubular conduit 120 comprises an outer diameter between approximately 4.2 mm and 5.1 mm, and/or a time period of approximately 13 minutes and 40 seconds when tubular conduit 120 comprises an outer diameter between approximately 5.2 mm and 6.0 mm.

Fiber matrix 110″ can comprise an average fiber size of approximately 8.6 μm, such as a population of fiber diameters with an average fiber size of approximately 8.6 μm with a standard deviation of 0.45 μm. Fiber matrix 110″ can comprise an average porosity of approximately 46.9%, such as a range of porosities with an average of 46.9% and a standard deviation of 0.9%. Fiber matrix 110″ can comprise a strength property selected from the group consisting of: stress at 5% strain comprising between 0.6 MPa and 1.3 MPa; ultimate stress of 5.0 MPa to 7.5MPa; ultimate strain of 200% to 400%; and combinations of these. Fiber matrix 110″ can comprise an average compliance (hereinafter “compliance”) between approximately 0.2×10⁻⁴/mmHg and 3.0×10⁻⁴/mmHg when measured in arterial pressure ranges. Fiber matrix 110″ can comprise an elastic modulus between 12 MPa and 18 MPa. Fiber matrix 110″ can be constructed and arranged with a targeted suture retention strength, such as an approximate suture retention strength of between 2.3 N and 4.3 N with 6-0 Prolene suture and/or between 2.0 N and 3.5 N with 7-0 Prolene suture. In some embodiments, graft device 100″ includes a spine 210, such as a spine 210 placed between inner and outer layers of fiber matrix 110″ (e.g. placed after one-third of the total thickness of fiber matrix 110″ is applied about conduit 120).

Fiber matrix 110″ of graft device 100″ can comprise more bonds between fibers than fiber matrix 110′ of graft device 100′. The increased number of bonds can result in a higher fiber matrix 110″ density which can be configured to limit cellular infiltration into graft device 100″ (e.g. to increase the graft durability in vivo). Fiber matrix 110″ can comprise fibers that are flatter (i.e. more oval versus round) and/or denser than fibers of fiber matrix 110′. Fiber matrix 110″ can have a greater resiliency than fiber matrix 110′.

Referring now to FIG. 4, a side sectional view of a portion of electrospinning device 400 of FIG. 3 is illustrated, consistent with the present inventive concepts. Electrospinning device 400 includes polymer delivery assembly 405 as has been described hereabove. Polymer delivery assembly 405 is operably attached to linear drive assembly 445, such as to allow reciprocating motion (in and out of the page). Polymer delivery assembly 405 includes nozzle 427, which is positioned in (e.g. fixed to) sleeve 406. Nozzle 427 is fluidly attached to delivery tube 425, such as to receive polymer material 111 of FIG. 3. Nozzle 427 is connected to a power supply, not shown but such as power supply 410 described hereabove in reference to FIG. 3. Surrounding nozzle 427 is a tube, sheath 407, which can also be positioned in (e.g. fixed to) sleeve 406. In some embodiments, a relatively continuous separation, gap 408, is positioned between the inner surface of sheath 407 and the outer surface of nozzle 427. Also shown in FIG. 4 is mandrel 250, which is surrounded by (i.e. has been inserted into) tubular conduit 120. At least a portion of a fiber matrix, inner layer 110a, has been applied circumferentially about tubular conduit 120. In a subsequent step, a spine can be applied about inner layer 110a, and/or an outer layer of fiber matrix can be applied about inner layer 110a.

In some embodiments, sleeve 406 is made of an electrically non-conductive material, such as an electrically non-conductive plastic such as polyoxymethylene (POM). Sleeve 406 can be constructed of electrically non-conductive materials to electrically isolate one or more components of polymer delivery assembly 405. Alternatively, sleeve 406 can comprise electrically conductive material, such as to apply a pre-determined electrical potential to sleeve 406 and/or to simplify electrical connection between one or more components of polymer delivery assembly 405, such as to simplify an electrical connection of nozzle 427 to a power supply of device 400. Similarly, sheath 407 can comprise an electrically conductive and/or an electrically non-conductive material. Sheath 407 can comprise a hypotube, such as a metal hypotube comprising the same material as nozzle 427 (e.g. stainless steel such as 403 stainless steel). Sheath 407 can be electrically connected with nozzle 427, such as via direct contact or via a wire, not shown. In some embodiments, a conductive sheath 407 that is electrically connected to nozzle 427 is constructed and arranged to limit inadvertent lateral motion of a delivered fiber stream and/or to reduce the likelihood of icicle formation (i.e. where the fiber streams wicks to the edge of nozzle 427 and forms potentially undesirable secondary streams of fiber). Alternatively, a non-conductive sheath 407 can be constructed and arranged to diminish the electrical field effect to the fiber stream while allowing for the collection of vapor in gap 408, which can prevent adverse effects on the stream as it spreads across the face of nozzle 427. Nozzle 427 can comprise a hypotube with a blunt distal end (e.g. a blunt end that is relatively orthogonal to the axis 428 of nozzle 427 and comprises minimal filleting or chamfering). Nozzle 427 can comprise a length of between 0.5 inches and 1.5 inches, such as a length of approximately 1.0 inches. In some embodiments, approximately 1 cm of nozzle 427 extends below sleeve 406. Nozzle 427 can comprise an ID between 0.014 inches and 0.018 inches, such as an ID of approximately 0.016 inches. Nozzle 427 can comprise a wall thickness of approximately 0.004 inches to 0.018 inches, such as a wall thickness of approximately 0.006 inches. In some embodiments, nozzle 427 comprises a wall with a stepped (e.g. multiple thickness) profile, such as a nozzle 427 with a thicker wall at its midsection than on its distal end.

Sheath 407 can be constructed and arranged to limit (e.g., eliminate or otherwise reduce) “icicle formation” during the electrospinning process. Icicles are secondary jets that form from the nozzle by several phenomenon, including solidified polymer solution, trapped gas bubbles, field instabilities or field disuniformities. For example, icicles can include polymer solution that is suspended (e.g., dripping or hanging) from the nozzle 427. The distal end of sheath 407 can be positioned flush (e.g. aligned) with the distal end of nozzle 427 as shown. The distal end of sheath 407 can comprise an end relatively perpendicular to the axis 428 of nozzle 427, such as a sharp and/or deburred end. In some embodiments, sheath 407 comprises an ID slightly greater than the OD of nozzle 427, such as to create a gap 408. In other embodiments, sheath 407 is in contact with nozzle 427, avoiding gap 408. In yet other embodiments, sheath 407 and nozzle 427 comprise a single component (e.g. a single, thick-walled tube). Sheath 407 can comprise an ID of approximately 0.08 inches and/or an OD of approximately 0.118 inches. Sheath 407 can comprise a wall thickness of between 0.025 inches and 0.085 inches, such as a wall thickness of approximately 0.055 inches. Sheath 407 can comprise a length between 12 mm and 20 mm, such as a length of approximately 16 mm.

In some embodiments, central axis 428 of nozzle 427 is relatively vertical, and perpendicular to central axis 435 of mandrel 250. Axis 428 of nozzle 427 can be offset from axis 435 of mandrel 250, such as an offset along a horizontal plane of approximately 0.3 cm to 2.0 cm, such as an offset of 0.5 cm to 0.8 cm. This horizontal offset, offset HOI shown, can be configured to limit (e.g. prevent) material provided to the nozzle 427 (e.g. polymer solution) from inadvertently being deposited (e.g. dripping due to gravity) onto the tubular conduit 120 or the fiber matrix 110.

In some embodiments, electrospinning device 400 includes one or more “object-free zones” (described hereabove in reference to FIG. 3) such as zones Z1, Z2 and Z3 shown in FIG. 4 and comprising one or more volumes of space that are absent of objects that could interfere with the electrospinning process of device 400. Zone Z1 comprises a cylindrical volume centered about axis 435 of mandrel 250. In some embodiments, zone Z1 comprises a diameter of between 5 cm and 15 cm, such as a diameter of approximately 10 cm. Zone Z1 can comprise a length approximating the length of tubular conduit 120 and/or mandrel 250. Zone Z2 comprises a cylindrical volume centered about axis 428 of nozzle 427. Zone Z2 extends below the distal end of nozzle 427 and comprises a diameter of between 5 cm and 15 cm, such as a diameter of approximately 10 cm. The object-free zones can take any shape, and can include one or more volumes of space positioned about nozzle 427 (e.g. zone Z1), about mandrel 250 (e.g. zone Z2) and/or about and including the volume of space between the surface of the distal end of nozzle 427 and the outer surface of mandrel 250 (e.g. zone Z3 as shown in FIG. 4). In some embodiments, object-free zones (e.g. Z1, Z2 and/or Z3) are sized and configured to be large enough to prevent one or more of: adversely affecting the electromagnetic field between nozzle 427 and mandrel 250; having an object interfere with (e.g. collide with) the flight path of a fiber traveling between nozzle 427 and mandrel 250; and allowing polymer material to drip onto tubular conduit 120 and/ro fiber matrix 110. In these embodiments, object-free zones Z1, Z2 and/or Z3 are of a small enough size to permit adequate desired deposition of fibers onto the tubular conduit 120 and/or the fiber matrix 110 during operation of device 400.

While the graft devices of the present invention have been described in detail as including an electrospun fiber matrix, other fiber delivery or other material application equipment can be used. The graft devices can include one or more spines, or the applied fiber matrix can be configured to sufficiently resist kinking without the inclusion of the spine.

While the preferred embodiments of the systems, methods and devices have been described in reference to the environment in which they were developed, they are merely illustrative of the principles of the inventions. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it can be possible, or even expedient in certain circumstances, to change the order in which some steps are performed, and it is intended that the particular steps of the method or procedure claim set forth herebelow not be construed as being order-specific unless such order specificity is expressly stated in the claim.

We claim:

1. A graft device for a mammalian patient, comprising:
a tubular conduit; and
a fiber matrix surrounding the tubular conduit;
wherein the device is constructed and arranged as described in at least one device claim herebelow.

2. The device of any device claim herein, wherein the fiber matrix comprises at least one thermoplastic co-polymer.

3. The device of claim 2, wherein the fiber matrix comprises a first material and a second different material.

4. The device of claim 3, wherein the second material comprises a softer material than the first material.

5. The device of claim 4, wherein the fiber matrix comprises relatively equal amounts of the first material and the second material.

6. The device of claim 4, wherein the second material comprises polydimethylsiloxane and a polyether-based polyurethane.

7. The device of claim 4, wherein the first material comprises aromatic methylene diphenyl isocyanate.

8. The device of any device claim herein, wherein the fiber matrix comprises a material selected from the group consisting of: polymer selected from the group consisting of: polyolefins; polyurethanes; polyvinylchlorides; polyamides; polyimides; polyacrylates; polyphenolics; polystyrene; polycaprolactone; polylactic acid; polyglycolic acid; and combinations thereof.

9. The device of claim 8, wherein the fiber matrix comprises a polymer applied to the tubular conduit when dissolved in a solvent.

10. The device of claim 9, wherein the solvent comprises a material selected from the group consisting of: hexafluoroisopropanol; acetone; methyl ethyl ketone; benzene; toluene; xylene; dimethyleformamide; dimethylacetamide; propanol; ethanol; methanol; propylene glycol; ethylene glycol; trichloroethane; trichloroethylene; carbon tetrachloride; tetrahydrofuran; cyclohexone; cyclohexpropylene glycol; DMSO; tetrahydrofuran; chloroform; methylene chloride; and combinations thereof.

11. The device of any device claim herein, wherein the fiber matrix comprises a material selected from the group consisting of: polyglycerol sebacate; hyaluric acid; silk fibroin collagen; elastin; poly(p-dioxanone); poly(3-hydroxybutyrate); poly(3-hydroxyvalerate); poly(valcrolactone); poly(tartronic acid); poly(beta-malonic acid); polypropylene fumarates); a polyanhydride; a tyrosine-derived polycarbonate; a polyorthoester; a degradable polyurethane; a polyphosphazene; and combinations thereof.

12. The device of any device claim herein, wherein the fiber matrix comprises a thermoplastic comprising at least two materials.

13. The device of claim 12, wherein the thermoplastic comprises a first material and a second material, and wherein the first material is softer than the second material.

14. The device of claim 13, wherein the first material comprises segments including polydimethylsiloxane and polyhexamethylene oxide, and the second material comprises segments including aromatic methylene diphenyl isocyanate.

15. The device of any device claim herein, wherein the fiber matrix comprises a material applied with a device selected from the group consisting of: an electrospinning device; a melt-spinning device; a melt-electrospinning device; a misting assembly; a sprayer; an electrosprayer; a three-dimensional printer; and combinations thereof.

16. The device of any device claim herein, wherein the fiber matrix comprises a thickness between 100 nm and 1000 nm.

17. The device of claim 16, wherein the fiber matrix comprises a thickness between 150 nm and 400 μm.

18. The device of claim 17, wherein the fiber matrix comprises a thickness between 220 nm and 280 nm.

19. The device of claim 17, wherein the fiber matrix comprises a thickness of approximately 250 μm.

20. The device of any device claim herein, wherein the fiber matrix comprises an inner layer and a surrounding outer layer.

21. The device of claim 20, further comprising a kink resisting element positioned between the inner layer and the outer layer.

22. The device of claim 21, wherein the kink resisting element comprises a spine.

23. The device of any device claim herein, wherein the fiber matrix comprises fibers with an average diameter of at least 5 μm.

24. The device of any device claim herein, wherein the fiber matrix comprises fibers with an average diameter between 6 μm and 15 μm.

25. The device of claim 24, wherein the fiber matrix comprises fibers with an average diameter of approximately 7.8 μm.

26. The device of claim 24, wherein the fiber matrix comprises fibers with an average diameter of approximately 8.6 μm.

27. The device of any device claim herein, wherein the fiber matrix comprises an average porosity between 40% and 80%.

28. The device of claim 27, wherein the fiber matrix comprises an average porosity of approximately 50.4%.

29. The device of claim 27, wherein the fiber matrix comprises an average porosity of approximately 46.9%.

30. The device of any device claim herein, wherein the fiber matrix comprises a compliance between 0.2×10⁻⁴/mmHg and 3.0×10⁻⁴/mmHg.

31. The device of any device claim herein, wherein the fiber matrix comprises an average elastic modulus between 10 MPa and 18 MPa.

32. The device of any device claim herein, wherein the fiber matrix comprises a property selected from the group consisting of: stress measured at 5% strain comprising between 0.4 MPa and 1.1 MPa; ultimate stress of 4.5 MPa to 7.0 MPa; ultimate strain of 200% to 400%; and combinations thereof.

33. The device of any device claim herein, wherein the fiber matrix comprises a property selected from the group consisting of: stress at 5% strain comprising between 0.6 MPa and 1.3 MPa; ultimate stress of 5.0 MPa to 7.5 MPa; ultimate strain of 200% to 400%; and combinations thereof.

34. The device of any device claim herein, wherein the fiber matrix comprises an average compliance of approximately 0.2×10⁴/mmHg to 3.0×10⁻⁴/mmHg.

35. The device of any device claim herein, wherein the fiber matrix comprises an average circumferential elastic modulus of between 10 MPa and 15 MPa.

36. The device of any device claim herein, wherein the fiber matrix comprises an average circumferential elastic modulus of between 12 MPa and 18 MPa.

37. The device of any device claim herein, wherein the fiber matrix is constructed and arranged to provide a suture retention strength of at least one of between 2.0 N and 4.0 N with 6-0 Prolene suture or between 1.5 N and 3.0 N with 7-0 Prolene suture.

38. The device of any device claim herein, wherein the fiber matrix is constructed and arranged to provide a suture retention strength of at least one of between 2.3 N and 4.3 N with 6-0 Prolene suture or between 2.0 N and 3.5 N with 7-0 Prolene suture.

39. The device of any device claim herein, further comprising a kink resisting element.

40. The device of claim 39, wherein the kink resisting element is positioned between the tubular conduit and the fiber matrix.

41. The device of claim 39, wherein the fiber matrix comprises an inner layer and an outer layer, and wherein the kink resisting element is positioned between the fiber matrix inner layer and outer layer.

42. The device of claim 41, wherein the fiber matrix comprises a first thickness and the inner layer comprises a second thickness approximately between 1% and 99% of the first thickness.

43. The device of claim 42, wherein the second thickness comprises a thickness approximately between 25% and 60% of the first thickness.

44. The device of claim 43, wherein the second thickness comprises a thickness of approximately 33% of the first thickness.

45. The device of claim 39, wherein the kink resisting element comprises a spine.

46. The device of claim 45, wherein the spine comprises a first support portion and a second support portion, wherein at least one of the first support portion or the second support portion is constructed and arranged to rotate relative to the other to receive the tubular conduit.

47. The device of claim 45, wherein the spine comprises a first support portion comprising a first set of projections, and a second support portion comprising a second set of projections, wherein the first set of projections interdigitate with the second set of projections.

48. The device of claim 47, wherein the interdigitating projections are spaced approximately 0.125 inches from each other.

49. The device of claim 47, wherein the interdigitating projections comprise a series of overlapping distal ends.

50. The device of claim 49, wherein the overlapping distal ends overlap at least 2.5 mm.

51. The device of claim 39, wherein the kink resisting element comprises at least one filament with a diameter between 0.4 mm and 0.5 mm.

52. The device of claim 39, wherein the kink resisting element comprises a resiliently biased element.

53. The device of claim 52, wherein the kink resisting element is resiliently biased with a heat treatment.

54. The device of claim 39, wherein the kink resisting element comprises a surface treated element.

55. The device of claim 54, wherein the kink resisting element surface treatment increases surface roughness of the kink resisting element.

56. The device of any device claim herein, wherein the device is constructed and arranged to be positioned in an in-vivo geometry including at least one arced portion comprising a radius of curvature of as low as 0.5 cm.

57. The device of any device claim herein, wherein the tubular conduit comprises a varying circumferential shape.

58. The device of any device claim herein, wherein the tubular conduit comprises harvested tissue.

59. The device of claim 58, wherein the tubular conduit comprises tissue selected from the group consisting of: saphenous vein; vein; artery; urethra; intestine; esophagus; ureter; trachea; bronchi; duct; fallopian tube; and combinations thereof.

60. The device of any device claim herein, wherein the tubular conduit comprises artificial material.

61. The device of claim 60, wherein the tubular conduit comprises artificial material selected from the group consisting of: polytetrafluoroethylene (PTFE); expanded PTFE (ePTFE); polyester; polyvinylidene fluoride/hexafluoropropylene (PVDF-HFP); silicone; polyethylene; polypropylene; polyester-based polymer; polyether-based polymer; thermoplastic rubber; and combinations thereof.

62. An system for applying a fiber matrix to a tubular conduit to create a graft device, the system comprising:
a rotating assembly constructed and arranged to rotate the tubular conduit;
a polymer delivery assembly constructed and arranged to deliver the fiber matrix to the tubular conduit; and
a controller constructed and arranged to control the polymer delivery assembly and the rotating assembly;
wherein the system is constructed and arranged as described in at least one system claim herebelow.

63. The system of any system claim herein, further comprising a polymer for delivery by the polymer delivery assembly.

64. The system of claim 63, wherein the polymer comprises at least one thermoplastic co-polymer.

65. The system of claim 63, wherein the polymer comprises at least two materials.

66. The system of claim 65, wherein the polymer comprises a first material and a second material, and wherein the first material is softer than the second material.

67. The device of claim 66, wherein the fiber matrix comprises relatively equal amounts of the first material and the second material.

68. The device of claim 66, wherein the first material comprises segments including polydimethylsiloxane and polyhexamethylene oxide, and the second material comprises segments including aromatic methylene diphenyl isocyanate.

69. The system of claim 63, further comprising a solvent combined with the polymer prior to application of the polymer to the tubular conduit.

70. The system of claim 69, wherein the solvent comprises HFIP.

71. The system of claim 70, wherein the solvent comprises 99.97% minimum purity HFIP.

72. The system of claim 69, wherein the solvent comprises a weight to volume ratio of concentration between 24.5 and 25.5% grams/mL.

73. The system of claim 63, wherein the polymer comprises molecular weight distribution between 80,000 and 150,000.

74. The system of claim 63, wherein the polymer comprises a viscosity between 2000 cP and 2400cP when measured at 25° C. and with a shear rate of 2020 s⁻¹.

75. The system of claim 63, wherein the polymer comprises a conductivity between 0.4 μS/cm and 1.7 μS/cm when measured at a temperature between 20° C. and 22° C.

76. The system of claim 63, wherein the polymer comprises a surface tension between 21.5 mN/m and 23.0 mN/m when measured at approximately 25° C.

77. The system of any system claim herein, wherein the polymer delivery assembly comprises an electrospinning device.

78. The system of any system claim herein, wherein the system is constructed and arranged to deliver polymer at a flow rate of between 10 ml/hr and 25 ml/hr.

79. The system of claim 78, wherein the system is constructed and arranged to deliver polymer at a flow rate of approximately 15 ml/hr.

80. The system of claim 78, wherein the system is constructed and arranged to deliver polymer at a flow rate of approximately 20 ml/hr.

81. The system of any system claim herein, wherein the polymer delivery assembly comprises a nozzle including an orifice constructed and arranged to deliver the fiber matrix to the tubular conduit.

82. The system of claim 81, wherein the system is constructed and arranged such that the tubular conduit is positioned approximately 12.2 cm and 12.8 cm from the orifice during application of the fiber matrix.

83. The system of claim 81, wherein the system is constructed and arranged to translate the nozzle a distance of approximately 29 cm in one direction.

84. The system of claim 81, wherein the system is constructed and arranged to translate the nozzle at a linear rate of between 40 mm/sec and 150 mm/sec.

85. The system of claim 84, wherein the system is constructed and arranged to translate the nozzle at a linear rate of between 50 mm/sec and 80 mm/sec.

86. The system of claim 84, wherein the system is constructed and arranged to translate the nozzle at a linear rate of between 55 mm/sec and 65 mm/sec.

87. The system of claim 84, wherein the system is constructed and arranged to translate the nozzle at a linear rate of approximately 60 mm/sec.

88. The system of claim 81, wherein the system is constructed and arranged to translate the nozzle in a reciprocal motion.

89. The system of claim 88, wherein the system is constructed and arranged to rapidly change direction at the end of each reciprocating linear travel.

90. The system of claim 81, wherein the system is constructed and arranged to apply a voltage of approximately +15 kV to the nozzle.

91. The system of claim 81, wherein the system is constructed and arranged to apply a voltage of approximately +17 kV to the nozzle.

92. The system of claim 81, wherein the nozzle comprises 304 stainless steel.

93. The system of claim 81, wherein the nozzle comprises a hypotube.

94. The system of claim 81, wherein the nozzle comprises a length of approximately 1.0 inches.

95. The system of claim 81, wherein the nozzle comprises an inner diameter of approximately 0.016 inches.

96. The system of claim 81, wherein the nozzle comprises a wall thickness between 0.004 inches and 0.018 inches.

97. The system of claim 96, wherein the nozzle comprises a wall thickness of approximately 0.006 inches.

98. The system of claim 81, wherein the nozzle comprises a central axis and a blunt end tip relatively orthogonal to the central axis.

99. The system of claim 81, wherein the system is constructed and arranged to maintain an object-free space distal to the orifice.

100. The system of claim 99, wherein the object-free space comprises a diameter of at least 10 cm.

101. The system of claim 81, wherein the nozzle comprises a central axis relatively perpendicular to a central axis of the tubular conduit during application of the fiber matrix.

102. The system of claim 81, wherein the nozzle comprises a proximal portion and wherein the polymer delivery assembly further comprises a sleeve surrounding the proximal portion of the nozzle.

103. The system of claim 102, wherein the sleeve comprises an insulating sleeve.

104. The system of claim 102, wherein the sleeve comprises a distal end positioned approximately 1 cm from the orifice.

105. The system of claim 81, wherein the nozzle is offset from a central axis of the tubular conduit during application of the fiber matrix.

106. The system of claim 105, wherein the offset comprises a distance of between 0.5 cm and 0.8 cm.

107. The system of claim 81, wherein the polymer delivery assembly further comprises a sheath surrounding the nozzle.

108. The system of claim 107, wherein the sheath is constructed and arranged to reduce icicle formation.

109. The system of claim 107, wherein the sheath comprises a distal end positioned flush with the orifice.

110. The system of claim 107, wherein the polymer delivery assembly comprises a gap positioned between the sheath and the nozzle.

111. The system of claim 107, wherein the sheath comprises a dimension selected from the group consisting of: length of approximately 16 mm; ID of approximately 0.08 inches; OD of approximately 0.118 inches; wall thickness between 0.025 inches and 0.085 inches; wall thickness approximately 0.055 inches; and combinations thereof.

112. The system of claim 107, wherein the nozzle comprises a proximal portion and wherein the polymer delivery assembly further comprises a sleeve surrounding the proximal portion of the nozzle.

113. The system of claim 112, wherein the sheath comprises a proximal portion and wherein the sleeve further surrounds the proximal portion of the sheath.

114. The system of any system claim herein, wherein the system is constructed and arranged to deliver the fiber matrix during a cumulative application time period.

115. The system of claim 114, wherein the cumulative application time period comprises approximately 11 minutes and 40 seconds for a fiber matrix with a diameter between 3.4 mm and 4.2 mm.

116. The system of claim 114, wherein the cumulative application time period comprises approximately 14 minutes and 0 seconds for a fiber matrix with a diameter between 4.2 mm and 5.1 mm.

117. The system of claim 114, wherein the cumulative application time period comprises approximately 17 minutes and 30 seconds for a fiber matrix with a diameter between 5.1 min and 6.0 mm.

118. The system of claim 114, wherein the cumulative application time period comprises approximately 9 minutes and 30 seconds for a fiber matrix with a diameter between 3.4 mm and 4.2 min.

119. The system of claim 114, wherein the cumulative application time period comprises approximately 11 minutes and 30 seconds for a fiber matrix with a diameter between 4.2 mm and 5.1 mm.

120. The system of claim 114, wherein the cumulative application time period comprises approximately 13 minutes and 40 seconds for a fiber matrix with a diameter between 5.1 mm and 6.0 mm.

121. The system of any system claim herein, wherein the polymer delivery assembly is constructed and arranged to deliver a fiber matrix comprising an inner layer and an outer layer.

122. The system of claim 121, wherein the system is constructed and arranged for a kink resisting element to be positioned between the inner layer and the outer layer.

123. The system of claim 121, wherein the system is constructed and arranged to deliver the fiber matrix during a cumulative application time period, and the inner layer is applied during approximately one-third of the cumulative application time period and the outer layer is applied during approximately two-thirds of the cumulative application time period.

124. The system of any system claim herein, further comprising a mandrel constructed and arranged for insertion into the tubular conduit.

125. The system of claim 124, wherein the system is constructed and arranged to apply a voltage of approximately −2 kV to the mandrel.

126. The system of claim 124, wherein the system is constructed and arranged to rotate the mandrel at a velocity between 100 rpm and 400 rpm.

127. The system of claim 126, wherein the system is constructed and arranged to rotate the mandrel at a velocity between 200 rpm and 300 rpm.

128. The system of claim 126, wherein the system is constructed and arranged to rotate the mandrel at a velocity between 240 pm and 260 rpm.

129. The system of claim 126, wherein the system is constructed and arranged to rotate the mandrel at a velocity of approximately 250 rpm.

130. The system of claim 124, wherein the mandrel comprises a material selected from the group consisting of: 304 stainless steel; 316 stainless steel; and combinations thereof.

131. The system of claim 124, wherein the mandrel comprises a surface finish of approximately Rₐ=0.1-0.8 μm.

132. The system of claim 124, wherein the mandrel comprises a length less than or equal to 45 cm.

133. The system of claim 124, wherein the mandrel comprises a length of at least 30 cm.

134. The system of claim 124, wherein the mandrel comprises one or more mandrels with a diameter selected from the group consisting of: 3.0 mm; 3.5 mm; 4.0 mm; 4.5 mm; and combinations thereof.

135. The system of claim 124, wherein the mandrel comprises a first mandrel and a second mandrel.

136. The system of claim 135, wherein the first mandrel comprises a first diameter and the second mandrel comprises a second diameter different than the first diameter.

137. The system of claim 124, wherein the mandrel comprises a mandrel central axis and the nozzle comprises a nozzle central axis positioned relatively orthogonal to the mandrel central axis during application of the fiber matrix.

138. The system of claim 124, wherein the mandrel comprises a mid portion and wherein the system is constructed and arranged to maintain an object-free space surrounding at least the mid portion of the nozzle.

139. The system of claim 138, wherein the object-free space comprises a diameter of at least 10 cm.

140. The system of any system claim herein, further comprising an environmental control assembly constructed and arranged to control at least one environmental parameter proximate the polymer delivery assembly and/or the tubular conduit during the application of the fiber matrix.

141. The system of claim 140, wherein the environmental control assembly comprises a component selected from the group consisting of: a fan; a source of a gas such as a dry compressed air source; a source of gas at a negative pressure; a vapor source such as a source including a buffered vapor, an alkaline vapor and/or an acidic vapor; a filter such as a HEPA filter; a dehumidifier; a humidifier; a heater; a chiller; and electrostatic discharge reducing ion generator; and combinations thereof.

142. The system of claim 140, wherein the at least one environmental parameter comprises humidity level.

143. The system of claim 140, wherein the environmental control assembly is constructed and arranged to provide flow of gas proximate the polymer delivery assembly and/or tubular conduit during application of the fiber matrix.

144. The system of claim 143, wherein the flow of gas comprises flow of gas comprising less than or equal to 2 ppm water content.

145. The system of claim 143, wherein the flow of gas is constructed and arranged to remove solvent vapors.

146. The system of claim 145, wherein the flow of gas is constructed and arranged to remove HFIP vapors.

147. The system of claim 143, wherein the flow of gas is constructed and arranged to replace gas surrounding the polymer delivery assembly and/or tubular conduit at least once every 3 minutes.

148. The system of claim 147, wherein the flow of gas is constructed and arranged to replace gas surrounding the polymer delivery assembly and/or tubular conduit at least once every 1 minute.

149. The system of claim 147, wherein the flow of gas is constructed and arranged to replace gas surrounding the polymer delivery assembly and/or tubular conduit at least once every 30 seconds.

150. The system of claim 143, wherein the environmental control assembly comprises a filter constructed and arranged to filter gas outflow from the environmental control assembly.

151. The system of claim 143, wherein the environmental control assembly is constructed and arranged to provide an initial flow of gas at a flow rate of at least 30 L/min.

152. The system of claim 151, wherein the environmental control assembly is constructed and arranged to provide an initial flow of gas at a flow rate of at least 60 L/min.

153. The system of claim 151, wherein the environmental control assembly is constructed and arranged to provide a reduced flow of gas after the initial flow of gas, and wherein the reduced flow of gas is provided at a flow rate between 10 L/min and 30 L/min.

154. The system of claim 140, wherein the at least one environmental parameter comprises a temperature level.

155. The system of claim 154, wherein the environmental control chamber is constructed and arranged to maintain a temperature between 15° C. and 25° C.

156. The system of claim 155, wherein the environmental control chamber is constructed and arranged to maintain a temperature between 16° C. and 20° C.

157. The system of claim 155, wherein the environmental control chamber is further constructed and arranged to maintain a relative humidity less than or equal to 24%.

158. The system of any system claim herein, further comprising a tubular conduit drying assembly.

159. The system of claim 158, wherein the tubular conduit drying assembly comprises gauze.

160. The system of any system claim herein, further comprising a modification assembly.

161. The system of claim 160, wherein the modification assembly comprises a component selected from the group consisting of: a robotic device such as a robotic device configured to apply a spine to the tubular conduit; a nozzle, such as a nozzle configured to deliver an agent; an energy delivery element such as a laser delivery element such as a laser excimer diode or other element configured to trim one or more components of the graft device; a fluid jet such as a water jet or air jet configured to deliver fluid during the application of the fiber matrix to the tubular conduit; a cutting element such as a cutting element configured to trim a spine and/or the fiber matrix; a mechanical abrader; and combinations thereof.

162. The system of claim 160, wherein the modification assembly is constructed and arranged to modify the tubular conduit.

163. The system of claim 160, wherein the modification assembly is constructed and arranged to modify the fiber matrix.

164. The system of claim 160, further comprising a kink resisting element, wherein the modification assembly is constructed and arranged to modify the kink resisting element.

165. The system of claim 160, further comprising a kink resisting element, wherein the modification assembly is constructed and arranged to apply the kink resisting element about the tubular conduit.

166. The system of claim 160, wherein the modification assembly is constructed and arranged to deliver an agent.

167. The system of claim 166, wherein the agent is constructed and arranged to be delivered to the tubular conduit to prevent damage to the tubular conduit by a solvent.

168. The system of claim 166, wherein the agent comprises an adhesive delivered between the tubular conduit and the fiber matrix.

169. The device of any device claim herein, wherein the fiber matrix comprises at least one thermoplastic co-polymer.

170. The device of claim 169, wherein the fiber matrix comprises a first material and a second different material.

171. The device of claim 170, wherein the second material comprises a softer material than the first material.

172. The device of claim 171, wherein the fiber matrix comprises relatively equal amounts of the first material and the second material.

173. The device of claim 171, wherein the second material comprises polydimethylsiloxane and a polyether-based polyurethane.

174. The device of claim 171, wherein the first material comprises aromatic methylene diphenyl isocyanate.

175. The device of any device claim herein, wherein the fiber matrix comprises a material selected from the group consisting of: polymer selected from the group consisting of: polyolefins; polyurethanes; polyvinylchlorides; polyamides; polyimides; polyacrylates; polyphenolics; polystyrene; polycaprolactone; polylactic acid; polyglycolic acid; and combinations thereof.

176. The device of claim 175, wherein the fiber matrix comprises a polymer applied to the tubular conduit when dissolved in a solvent.

177. The device of claim 176, wherein the solvent comprises a material selected from the group consisting of: hexafluoroisopropanol; acetone; methyl ethyl ketone; benzene; toluene; xylene; dimethyleformamide; dimethylacetamide; propanol; ethanol; methanol; propylene glycol; ethylene glycol; trichloroethane; trichloroethylene; carbon tetrachloride; tetrahydrofuran; cyclohexone; cyclohexpropylene glycol; DMSO; tetrahydrofuran; chloroform; methylene chloride; and combinations thereof.

178. The device of any device claim herein, wherein the fiber matrix comprises a material selected from the group consisting of: polyglycerol sebacate; hyaluric acid; silk fibroin collagen; elastin; poly(p-dioxanone); poly(3-hydroxybutyrate); poly(3-hydroxyvalerate); poly(valcrolactone); poly(tartronic acid); poly(beta-malonic acid); poly(propylene fumarates); a polyanhydride; a tyrosine-derived polycarbonate; a polyorthoester; a degradable polyurethane; a polyphosphazene; and combinations thereof.

179. The device of any device claim herein, wherein the fiber matrix comprises a thermoplastic comprising at least two materials.

180. The device of claim 179, wherein the thermoplastic comprises a first material and a second material, and wherein the first material is softer than the second material.

181. The device of claim 180, wherein the first material comprises segments including polydimethylsiloxane and polyhexamethylene oxide, and the second material comprises segments including aromatic methylene diphenyl isocyanate.

182. The device of any device claim herein, wherein the fiber matrix comprises a material applied with a device selected from the group consisting of: an electrospinning device; a melt-spinning device; a melt-electrospinning device; a misting assembly; a sprayer; an electrosprayer; a three-dimensional printer; and combinations thereof.

183. The device of any device claim herein, wherein the fiber matrix comprises a thickness between 100 μm and 1000 μm.

184. The device of claim 183, wherein the fiber matrix comprises a thickness between 150 μm and 400 μm.

185. The device of claim 184, wherein the fiber matrix comprises a thickness between 220 μm and 280 μm.

186. The device of claim 184, wherein the fiber matrix comprises a thickness of approximately 250 m.

187. The device of any device claim herein, wherein the fiber matrix comprises an inner layer and a surrounding outer layer.

188. The device of claim 187, further comprising a kink resisting element positioned between the inner layer and the outer layer.

189. The device of claim 188, wherein the kink resisting element comprises a spine.

190. The device of any device claim herein, wherein the fiber matrix comprises fibers with an average diameter of at least 5 μm.

191. The device of any device claim herein, wherein the fiber matrix comprises fibers with an average diameter between 6 μm and 15 μm.

192. The device of claim 191, wherein the fiber matrix comprises fibers with an average diameter of approximately 7.8 μm.

193. The device of claim 191, wherein the fiber matrix comprises fibers with an average diameter of approximately 8.6 μm.

194. The device of any device claim herein, wherein the fiber matrix comprises an average porosity between 40% and 80%.

195. The device of claim 194, wherein the fiber matrix comprises an average porosity of approximately 50.4%.

196. The device of claim 194, wherein the fiber matrix comprises an average porosity of approximately 46.9%.

197. The device of any device claim herein, wherein the fiber matrix comprises a compliance between 0.2×10⁻⁴/mmHg and 3.0×10⁻⁴/mmHg.

198. The device of any device claim herein, wherein the fiber matrix comprises an average elastic modulus between 10 MPa and 18 MPa.

199. The device of any device claim herein, wherein the fiber matrix comprises a property selected from the group consisting of: stress measured at 5% strain comprising between 0.4 MPa and 1.1 MPa; ultimate stress of 4.5 MPa to 7.0 MPa; ultimate strain of 200% to 400%; and combinations thereof.

200. The device of any device claim herein, wherein the fiber matrix comprises a property selected from the group consisting of: stress at 5% strain comprising between 0.6 MPa and 1.3 MPa; ultimate stress of 5.0 MPa to 7.5 MPa; ultimate strain of 200% to 400%; and
combinations thereof.

201. The device of any device claim herein, wherein the fiber matrix comprises an average compliance of approximately 0.2×10⁻⁴/mmHg to 3.0×10⁻⁴/mmHg.

202. The device of any device claim herein, wherein the fiber matrix comprises an average circumferential elastic modulus of between 10 MPa and 15 MPa.

203. The device of any device claim herein, wherein the fiber matrix comprises an average circumferential elastic modulus of between 12 MPa and 18 MPa.

204. The device of any device claim herein, wherein the fiber matrix is constructed and arranged to provide a suture retention strength of at least one of between 2.0 N and 4.0 N with 6-0 Prolene suture or between 1.5 N and 3.0 N with 7-0 Prolene suture.

205. The device of any device claim herein, wherein the fiber matrix is constructed and arranged to provide a suture retention strength of at least one of between 2.3 N and 4.3 N with 6-0 Prolene suture or between 2.0 N and 3.5 N with 7-0 Prolene suture.

206. The system of any system claim herein, further comprising a kink resisting element.

207. The device of claim 206, wherein the kink resisting element is positioned between the tubular conduit and the fiber matrix.

208. The device of claim 206, wherein the fiber matrix comprises an inner layer and an outer layer, and wherein the kink resisting element is positioned between the fiber matrix inner layer and outer layer.

209. The device of claim 208, wherein the fiber matrix comprises a first thickness and the inner layer comprises a second thickness approximately between 1% and 99% of the first thickness.

210. The device of claim 209, wherein the second thickness comprises a thickness approximately between 25% and 60% of the first thickness.

211. The device of claim 210, wherein the second thickness comprises a thickness of approximately 33% of the first thickness.

212. The device of claim 206, wherein the kink resisting element comprises a spine.

213. The device of claim 212, wherein the spine comprises a first support portion and a second support portion, wherein at least one of the first support portion or the second support portion is constructed and arranged to rotate relative to the other to receive the tubular conduit.

214. The device of claim 212, wherein the spine comprises a first support portion comprising a first set of projections, and a second support portion comprising a second set of projections, wherein the first set of projections interdigitate with the second set of projections.

215. The device of claim 214, wherein the interdigitating projections are spaced approximately 0.125 inches from each other.

216. The device of claim 214, wherein the interdigitating projections comprise a series of overlapping distal ends.

217. The device of claim 216, wherein the overlapping distal ends overlap at least 2.5 mm.

218. The device of claim 206, wherein the kink resisting element comprises at least one filament with a diameter between 0.4 mm and 0.5 mm.

219. The device of claim 206, wherein the kink resisting element comprises a resiliently biased element.

220. The device of claim 219, wherein the kink resisting element is resiliently biased with a heat treatment.

221. The device of claim 206, wherein the kink resisting element comprises a surface treated element.

222. The device of claim 221, wherein the kink resisting element surface treatment increases surface roughness of the kink resisting element.

223. The device of any device claim herein, wherein the device is constructed and arranged to be positioned in an in-vivo geometry including at least one arced portion comprising a radius of curvature of as low as 0.5 cm.

224. A method as described in reference to the figures.

225. A method of creating a graft device as described in reference to the figures.

226. A system for creating a graft device as described in reference to the figures.

227. A graft device as described in reference to the figures.
